(19) United States
(12) Patent Application Publication
(10) Pub. No.: US 2022/0282247 A1
(43) Pub. Date: Sep. 8, 2022

(54) EXON 44-TARGETED NUCLEIC ACIDS AND RECOMBINANT ADENO-ASSOCIATED VIRUS COMPRISING SAID NUCLEIC ACIDS FOR TREATMENT OF DYSTROPHIN-BASED MYOPATHIES

(71) Applicant: RESEARCH INSTITUTE AT NATIONWIDE CHILDREN'S HOSPITAL, Columbus, OH (US)

(72) Inventors: Nicolas Sebastien Wein, Columbus, OH (US); Kevin Flanigan, Columbus, OH (US)

(21) Appl. No.: 17/632,263

(22) PCT Filed: Aug. 3, 2020
(86) PCT No.: PCT/US20/44755

§ 371 Date: Feb. 2, 2022

Related U.S. Application Data
(60) Provisional application No. 62/882,216, filed on Aug. 2, 2019.

(51) Int. Cl. C12N 15/113; A61P 21/00

(52) U.S. Cl. CPC C12N 15/113; A61P 21/00; C12N 2320/33; C12N 2310/11

(57) ABSTRACT

The disclosure relates to the field of gene therapy for the treatment of a muscular dystrophy including, but not limited to, Duchenne Muscular Dystrophy (DMD). More particularly, the disclosure provides nucleic acids, including nucleic acids encoding U7-based small nuclear ribonucleic acids (RNAs) (snRNAs), U7-based snRNAs, and recombinant adeno-associated virus (rAAV) comprising the nucleic acid molecules to deliver nucleic acids encoding U7-based snRNAs to induce exon-skipping for use in treating a muscular dystrophy including, but not limited to, DMD, resulting from a mutation amenable to skipping exon 44 of the DMD gene (DMD exon 44) including, but not limited to, any mutation involving, surrounding, or affecting DMD exon 44.

### CROSS-REFERENCE TO RELATED APPLICATIONS

This application claims the benefit of prior U.S. provisional application No. 62/882,216, filed Aug. 2, 2018, the disclosure of which is incorporated by reference in its entirety

### FIELD

The disclosure relates to the field of gene therapy for the treatment of muscular dystrophy. More particularly, the disclosure provides nucleic acids, including nucleic acids encoding U7-based small nuclear ribonucleic acids (RNAs) (snRNAs), U7-based snRNAs, and recombinant adeno-associated virus (rAAV) comprising the nucleic acid molecules to deliver nucleic acids encoding U7-based snRNAs to induce exon-skipping for use in treating a muscular dystrophy resulting from a mutation amenable to skipping exon 44 of the DMD gene (DMD exon 44) including, but not limited to, any mutation involving, surrounding, or affecting DMD exon 44.

### INCORPORATION BY REFERENCE OF THE SEQUENCE LISTING

This application contains, as a separate part of disclosure, a Sequence Listing in computer-readable form (filename: 54313A_Seqlisting.txt; Size: 22,771 bytes: Created: Aug. 3, 2020) which is incorporated by reference herein in its entirety.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). It is the most common severe childhood form of muscular dystrophy affecting 1 in 5000 newborn males. DMD is caused by mutations in the DMD gene leading to absence of dystrophin protein (427 KDa) in skeletal and cardiac muscles, as well as the gastrointestinal tract and retina. Dystrophin not only protects the sarcolemma from eccentric contractions, but also anchors a number of signaling proteins in close proximity to sarcolemma. Another form of MD is Becker Muscular Dystrophy (BMD). BMD, like DMD, is a genetic disorder that gradually makes the body's muscles weaker and smaller. BMD affects the muscles of the hips, pelvis, thighs, and shoulders, as well as the heart, but is known to cause less severe problems than DMD.

Many clinical cases of DMD are linked to deletion mutations in the DMD gene. In contrast to the deletion mutations, DMD exon duplications account for around 5% of disease-causing mutations in unbiased samples of dystrophinopathy patients [Dent et al., Am J Med Genet, 134(3): 295-298 (2005)], although in some catalogues of mutations the number of duplications is higher, including that published by the United Dystrophinopathy Project by Flanigan et al. [Hum Mutat, 30(12): 1657-1666 (2009)], in which it was 11%. BMD is also caused by a change in the dystrophin gene, which makes the protein too short. The flawed dystrophin puts muscle cells at risk for damage with normal use. See also, U.S. Patent Application Publication Nos. 2012/0077860, published Mar. 29, 2012; 2013/0072541, published Mar. 21, 2013; and 2013/0045538, published Feb. 21, 2013.

A deletion of exon 45 is one of the most common deletions found in DMD patients, whereas a deletion of exons 44 and 45 is generally associated with BMD [Anthony et al., JAMA Neurol 71:32-40 (2014)]. Thus, if exon 44 could be bypassed in pre-messenger RNA (mRNA), transcripts of these DMD patients, this would restore the reading frame and enable the production of a partially functional BMD-like dystrophin [Aartsma-Rus et al., Nucleic Acid Ther 27(5): 251-259 (2017)]. In fact, it appears that many patients with a deletion bordering on exon 45, skip exon 44 spontaneously, although at very low levels. This results in slightly increased levels of dystrophin when compared with DMD patients carrying other deletions, and most likely underlies the less severe disease progression observed in these patients compared with DMD patients with other deletions [Anthony et al., supra; Pane et al., PLoS One 9:e83400 (2014); van den Bergen et al., J Neuromuscul Dis 1:91-94 (2014)].

Despite many lines of research following the identification of the DMD gene, treatment options are limited. There thus remains a need in the art for treatments for MDs, including DMD. The most advanced therapies include those that aim at restoration of the missing protein, dystrophin, using mutation-specific genetic approaches, such as antisense oligonucleotide (AON)-mediated exon skipping.

### SUMMARY

The disclosure provides products, methods, and uses for a new gene therapy for treating, ameliorating, delaying the progression of, and/or preventing a muscular dystrophy involving a mutation amenable to skipping exon 44 of the DMD gene (DMD exon 44) including, but not limited to, any mutation involving, surrounding, or affecting DMD exon 44. More particularly, the disclosure provides nucleic acids, U7-based small nuclear ribonucleic acids (RNAs) (snRNAs), and recombinant adeno-associated virus (rAAV) comprising the nucleic acid molecules to deliver nucleic acids encoding U7-based snRNAs to induce exon-skipping to provide an altered form of dystrophin protein for use in treating a muscular dystrophy resulting from a duplication of DMD exon 44, a deletion of exon 43 or 45, or a deletion of exons 45-56.

The disclosure provides a nucleic acid molecule that binds or is complementary to a polynucleotide encoding exon 44 of the DMD gene, wherein the polynucleotide encoding DMD exon 44 comprises or consists of the nucleotide sequence set out in SEQ ID NO: 1 or 2 or encodes the amino acid sequence set out in SEQ ID NO: 3.

The disclosure provides a nucleic acid molecule that binds or is complementary to at least one of the nucleotide sequences set out in SEQ ID NO: 4, 5, 6, 7, 32, 33, 34, or 35.

The disclosure provides a nucleic acid molecule comprising or consisting of a nucleotide sequence having at least 80%, at least 85%, at least 90%, at least 95%, at least 96%, at least 97%, at least 98%, or at least 99% identity to the nucleotide sequence set out in SEQ ID NO: 4, 5, 6, 7, 8, 9, 10, 11, 12, 13, 14, 15, 32, 33, 34, or 35. The disclosure provides a nucleic acid molecule comprising or consisting of the nucleotide sequence set out in SEQ ID NO: 4, 5, 6, 7, 8, 9, 10, 11, 12, 13, 14, 15, 32, 33, 34, or 35.

The disclosure provides a nucleic acid molecule comprising or consisting of a nucleotide sequence having at least 80%, at least 85%, at least 90%, at least 95%, at least 96%, at least 97%, at least 98%, or at least 99% identity to the nucleotide sequence set out in SEQ ID NO: 16, 17, 18, 19, 20, 21, 22, 23, 24, 25, 26, or 27. The disclosure provides a nucleic acid molecule comprising or consisting of the nucleotide sequence set out in SEQ ID NO: 16, 17, 18, 19, 20, 21, 22, 23, 24, 25, 26, or 27.

The disclosure provides a recombinant adeno-associated virus (rAAV) comprising a genome comprising at least one of the nucleic acid molecules disclosed or described herein. In some aspects, the disclosure provides an rAAV, wherein the genome of the rAAV is a self-complementary genome or a single-stranded genome. In some aspects, the rAAV is rAAV-1, rAAV-2, rAAV-3, rAAV-4, rAAV-5, rAAV-6, rAAV-7, rAAV-8, rAAV-9, rAAV-10, rAAV-11, rAAV-12, rAAV-13, rAAV-rh74, or rAAV-anc80. In some aspects, the disclosure provides an rAAV, wherein the genome of the rAAV lacks AAV rep and cap DNA. In some aspects, the disclosure provides an rAAV, wherein the rAAV further comprises an AAV-1 capsid, an AAV-2 capsid, an AAV-3 capsid, an AAV-4 capsid, an AAV-5 capsid, an AAV-6 capsid, an AAV-7 capsid, an AAV-8 capsid, an AAV-9 capsid, an AAV-10 capsid, an AAV-11 capsid, an AAV-12 capsid, an AAV-13 capsid, an AAV-rh74 capsid, or an AAV-anc80 capsid.

The disclosure provides methods for inducing skipping of exon 44 of the DMD gene in a cell. In some aspects, the methods comprise providing the cell with at least one of the nucleic acid molecules disclosed or described herein. In some aspects, the methods comprise providing the cell with more than one of the nucleic acid molecules disclosed or described herein. In some aspects, the methods comprise provide the cell with an rAAV comprising at least one of the nucleic acid molecules disclosed or described herein. In some aspects, the methods comprise provide the cell with an rAAV comprising more than one of the nucleic acid molecules disclosed or described herein.

The disclosure provides methods for treating, ameliorating, and/or preventing a muscular dystrophy in a subject with any mutation amenable to DMD exon 44 skipping comprising administering to the subject at least one of the nucleic acid molecules disclosed or described herein. In some aspects, the methods comprise administering to the subject an rAAV comprising at least one of the nucleic acid molecules disclosed or described herein. In some aspects, the methods comprise administering to the subject an rAAV comprising more than one of the nucleic acid molecules disclosed or described herein. In some aspects, the mutation amenable to DMD exon 44 skipping is a mutation in the DMD gene sequence involving, surrounding, or affecting DMD exon 44. In some aspects, the mutation is a deletion of exons 1-43, 2-43, 3-43, 4-43, 5-43, 6-43, 7-43, 8-43, 9-43, 10-43, 11-43, 12-43, 13-43, 14-43, 15-43, 16-43, 17-43, 18-43, 19-43, 20-43, 21-43, 22-43, 23-43, 24-43, 25-43, 26-43, 27-43, 28-43, 29-43, 30-43, 31-43, 32-43, 33-43, 34-43, 35-43, 36-43, 37-43, 38-43, 39-43, 40-43, 41-43, 42-43, 43, 45, 45-46, 45-47, 45-48, 45-49, 45-50, 45-51, 45-52, 45-53, 45-54, 45-55, 45-56, 45-57, 45-58, 45-59, 45-60, 45-61, 45-62, 45-63, 45-64, 45-65, 45-66, 45-67, 45-68, 45-69, 45-70, 45-71, 45-72, 45-73, 45-74, 45-75, 45-76, 45-77, and 45-78, and/or a duplication of exon 44. In some aspects, the mutation is a duplication of DMD exon 44, a deletion of exon 43 or 45, or a deletion of exons 45-56. In some aspects, the administering results in increased expression of dystrophin protein including, but not limited to, increased expression of an altered form of dystrophin protein or a functionally active altered form or fragment of dystrophin protein in the subject. In some aspects, the administering inhibits the progression of dystrophic pathology in the subject. In some aspects, the administering improves muscle function in the subject. In some aspects, such improvement in muscle function is an improvement in muscle strength. In some aspects, such improvement in muscle function is an improvement in stability in standing and walking.

The disclosure provides the use of at least one of the nucleic acid molecules disclosed or described herein for inducing skipping of exon 44 of the DMD gene in a cell. In some aspects, the cell is found within a subject or is isolated from a subject with a mutation involving, surrounding, or affecting DMD exon 44. In some aspects, the nucleic acid molecules are provided in an rAAV. In some aspects, more than one of the various nucleic acid molecules disclosed or described herein or a combination of the various nucleic acid molecules disclosed or described herein are provided in an rAAV.

The disclosure provides the use of at least one of the nucleic acid molecules disclosed or described herein in treating, ameliorating, and/or preventing a muscular dystrophy in a subject with a mutation involving, surrounding, or affecting DMD exon 44. The disclosure includes the use of at least one of the nucleic acid molecules disclosed or described herein in the preparation of a medicament for treating, ameliorating, and/or preventing a muscular dystrophy in a subject with a mutation involving, surrounding, or affecting DMD exon 44. In some aspects, the nucleic acid molecules are provided in an rAAV. In some aspects, more than one of the various nucleic acid molecules disclosed or described herein or a combination of the various nucleic acid molecules disclosed or described herein are provided in an rAAV. In some aspects, the mutation is a mutation in the sequence involving, surrounding, or affecting DMD exon 44. In some aspects, the mutation is a duplication of DMD exon 44, a deletion of exon 43 or 45, or a deletion of exons 45-56. In some aspects, the use results in increased expression of dystrophin protein or increased expression of an altered form of dystrophin protein which has functional activity of the dystrophin protein. In some aspects, the use inhibits the progression of dystrophic pathology. In some aspects, the use improves muscle function. In some aspects, the improvement in muscle function is an improvement in muscle strength. In some aspects, the improvement in muscle function is an improvement in stability in standing and walking.

Other features and advantages of the disclosure will become apparent from the following description of the drawing and the detailed description. It should be understood, however, that the drawing, detailed description, and the specific examples, while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWING

FIG. 1A-F shows exon skipping of human DMD exon 44 after transduction of Del45-56 FibroMyoD, Del45 FibroMyoD, and Dup44 FibroMyoD with various viral constructs. FIG. 1A shows results of RT-PCR of Del45-56 FibroMyoD treated with SD44, LESE44, or SESE44 constructs [Del45-56 (untreated) and Del 44-56 (treated)]. Del45-56 FibroMyoD treated with SD44 exhibit exon skipping as shown by the strong band in Del44-56. Del45-56 FibroMyoD treated with LESE44 or SESE44 exhibit partial exon skipping as shown by bands in Del45-56 and Del44-56. FIG. 1B shows RT-PCR of Del45 FibroMyoD treated with LESE44, SESE44, SD44, and BP43AS44 constructs [Del45 (untreated) and Del 44-45 (treated)]. Although all treated FibroMyoD exhibit exon skipping, SD44 shows the greatest amount of exon skipping. FIG. 1C shows RT-PCR of Dup44 FibroMyoD treated with SD44, BP43AS44, and LESE44 constructs [Del45 (untreated) and Del 44-45 (treated)]. Although all treated FibroMyoD exhibit exon skipping, SD44 appears to show the greatest amount of exon skipping. FIG. 1D shows results of RT-PCR of Del45-56 FibroMyoD treated with SD44, 4X-SD44, or SD44-stuffer constructs [Del45-56 (untreated) and Del 44-56 (treated)]. Del45-56 FibroMyoD treated with all constructs show strong exon skipping as shown by the strong band in Del44-56 in all three constructs, with the most intense bands found in FibroMyoD treated with 4X-SD44 and SD44-stuffer constructs. FIG. 1E shows RT-PCR of Del45 FibroMyoD treated with 4X-SD44, SD44-stuffer, and SD44 constructs [Del45 (untreated) and Del 44-45 (treated)]. All treated FibroMyoD exhibit strong exon 44 skipping in Del45 FibroMyoD. FIG. 1E shows RT-PCR of Dup44 FibroMyoD treated with SD44-stuffer, 4X-SD44, and SD44 constructs [Del45 (untreated) and Del 44-45 (treated)]. All treated FibroMyoD exhibit strong exon skipping, with both SD44-stuffer and 4X-SD44 showing the greatest amount of exon skipping in these experiments.

FIG. 2 shows the efficient skipping of human DMD exon 44 in the tibialis anterior (TA) muscle of 3-month old hDMDdel45/mdx mice, one month after injection with the three different rAAV viral vectors. Experiments were performed in each TA of two mice (n=4 TA muscles per construct). These RT-PCR results demonstrated absence of exon skipping in mice #57 and #58 (untreated hDMDdel45/mdx mice); efficient exon skipping in mice #60 and #61 (hDMDdel45/mdx mice injected with U7-SD44-stuffer (SEQ ID NO: 27); efficient exon skipping in mice #66 and #72 (hDMDdel45/mdx mice injected with U7-SD44 (SEQ ID NO: 23)); and efficient exon skipping in mouse #84 (hDMDdel45/mdx mouse injected with U7-4x-SD44 (SEQ ID NO: 26)). Black 6 (Bl6) mouse is a wild-type mouse that does not contain the human DMD gene and, therefore, is a negative control for human DMD.

FIG. 3A-E shows the immunofluorescent expression of human dystrophin in the tibialis anterior (TA) muscle of 3-month old hDMD/mdx del45 mice, one month after injection with the three different rAAV viral vectors. Experiments were performed in each TA of two mice (n=4 TA muscles per construct). These immunofluorescence results were obtained from #58 (untreated mice); from mouse #72 (mouse injected with U7-SD44 (SEQ ID NO: 23); FIG. 3C); from mouse #60 (mouse injected with U7-SD44-stuffer (SEQ ID NO: 27); FIG. 3D); and from mouse #84 (mouse injected with U7-4x-SD44 (SEQ ID NO: 26); FIG. 3E). Bl6 is a wild type mouse that does not contain the human DMD gene but the antibody used in this immunofluorescence experiment recognizes both human and mouse dystrophin. After one month of treatment, immunostaining indicates that dystrophin was expressed after viral infection with all three rAAV viral vectors, with the SD44-stuffer vector (FIG. 3D) and the 4X-SD44 vector (FIG. 3E) appearing to result in the greatest level of dystrophin expression in the muscle. FIG. 3A shows no dystrophin expression in the untreated hDMDdel45/mdx mouse. FIG. 3B shows dystrophin expression in the Bl6 model because the antibody reacts with mouse dystrophin.

FIG. 4 shows Western blot expression of human dystrophin in the tibialis anterior (TA) muscle of hDMD/mdx del45 mice one month after injection with the three different rAAV viral vectors. Experiments were performed in each TA of two mice (n=4 TA muscles per construct). After one month, Western blots result show that dystrophin was expressed after infection with all three rAAV viral vectors, with the SD44stuffer vector appearing to result in the greatest level of dystrophin expression in the muscle. These Western blot results were obtained from mice #57 and #58 (untreated hDMD/mdx del45 mice); from mice #60 and #61 (hDMD/mdx del45 mice injected with U7-SD44-stuffer (SEQ ID NO: 27)); from mice #66 and #72 (hDMD/mdx del45 mice injected with U7-SD44 (SEQ ID NO: 23)) and from mouse #84 (hDMD/mdx del45 mouse injected with U7-4x-SD44 (SEQ ID NO: 26)). Bl6 is a wild type mouse that does not contain the human DMD gene; however, the antibody used in this Western blot recognizes both human and mouse dystrophin. Actinin was used a control.

FIG. 5A-E shows efficient exon skipping of human DMD exon 44 after transduction of hDMD/mdx del45 mice three months post injection, protein restoration and muscle force improvement. FIG. 5A shows results of RT-PCR of hDMD/mdx del45 mice. FIG. 5A shows the efficient skipping of human DMD exon 44 in the tibialis anterior (TA) muscle of 3-month old hDMDdel45/mdx mice, three months after injection with the rAAV.U7_SD44stuffer viral vector. Experiments were performed in each tibialis anterior (TA) of two mice (n=6 TA muscles). These RT-PCR results demonstrated very rare exon skipping in mice (untreated hDMDdel45/mdx mice n=6 TA muscles); and efficient exon skipping in mice (hDMDdel45/mdx mice injected with rAAV.U7_SD44stuffer (n=6 TA muscles; SEQ ID NO: 27). WT mouse is a wild-type mouse that does not contain the human DMD gene, but contains the mouse DMD gene; therefore, this WT mouse is a positive control. FIG. 5B shows Western blot expression of human dystrophin in the TA muscle of hDMD/mdx del45 mice three month after injection with rAAV.U7_SD44stuffer. Experiments were performed in each TA of three mice (n=6 TA muscles). After three months, Western blots result showed that dystrophin was expressed after infection with with the rAAV.U7_SD44stuffer (SEQ ID NO: 27). These Western blot results were obtained from mice, i.e., 3 out of the 6 TA injected). WT is a wild type mouse that does not contain the human DMD gene; however, the antibody used in this Western blot recognizes both human and mouse dystrophin. Actinin was used a control. FIGS. 5C-E show improvement of muscle force three months post-injection with rAAV.U7_SD44stuffer (SEQ ID NO: 27). FIG. 5C shows improvement of the hang wire; FIG. 5D shows specific force; and FIG. 5E shows eccentric contraction three months post-injection.

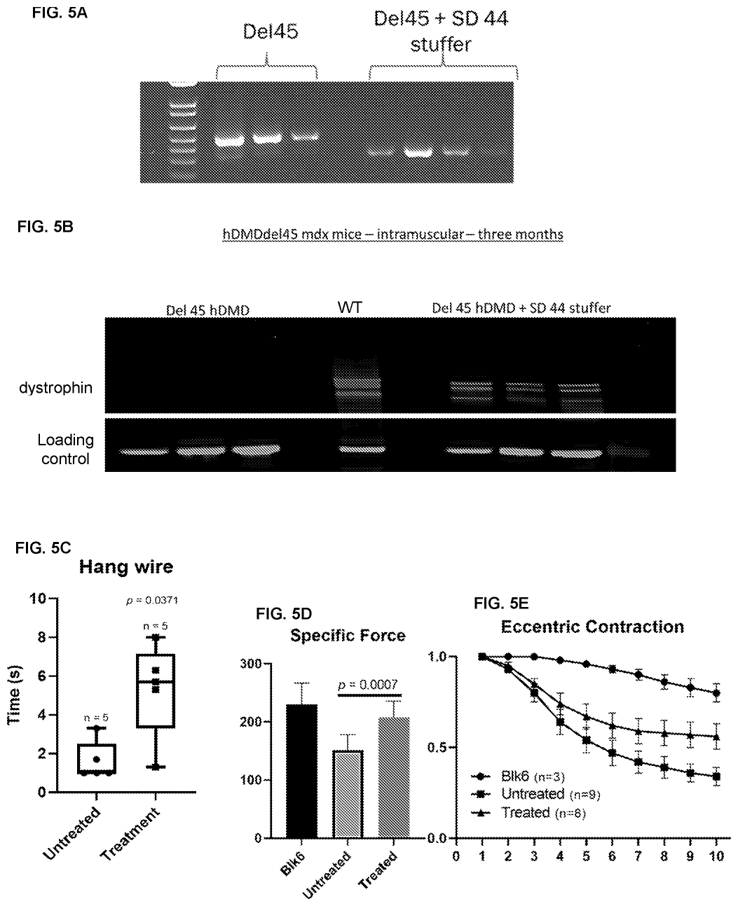

### DETAILED DESCRIPTION

The disclosure provides products, methods, and uses for treating, ameliorating, delaying the progression of, and/or preventing a muscular dystrophy involving a mutation involving, surrounding, or affecting DMD exon 44, including but not limited to, a duplication of DMD exon 44, a deletion of exon 43 or 45, or a deletion of exons 45-56. DMD, the largest known human gene, provides instructions for making a protein called dystrophin. Dystrophin is located primarily in muscles used for movement (skeletal muscles) and in heart (cardiac) muscle.

More particularly, the disclosure provides nucleic acids comprising sequences designed to bind DMD exon 44 or DMD exon 44 and its surrounding intronic sequence to provide an altered form of dystrophin protein for use in treating a muscular dystrophy resulting from a mutation involving, surrounding, or affecting DMD exon 44. The disclosure provides nucleic acids comprising nucleotide sequences encoding and comprising U7-based small nuclear ribonucleic acids (snRNAs) (U7 snRNAs), and vectors, such as recombinant adeno-associated virus (rAAV), comprising the nucleic acids to deliver nucleic acids encoding U7-based snRNAs to induce exon-skipping of DMD exon 44 to provide an altered form of dystrophin protein for use in treating a muscular dystrophy resulting from a mutation involving, surrounding, or affecting DMD exon 44. Exon skipping is a treatment approach to correct and restore production of dystophin. For specific genetic mutations it allows the body to make a shorter, usable dystophin. Although up to now exon skipping is not a cure for DMD, it may make the effects of DMD less severe.

Thus, the disclosure provides nucleic acids for treating any mutation amenable to exon 44 skipping. In some aspects, such mutation amenable to exon 44 skipping is a mutation involving, surrounding, or affecting DMD exon 44. Examples of such mutations amenable to exon 44 skipping include, but are not limited to, those provided at https colon-slash-slash-www.cureduchenne.org-slash-wp-content-slash-uploads-slash-2016-slash-11-slash-Duchenne-Population-Potentially-Amenable-to-Exon-Skipping-11.10.16.pdf. Such exon 44 skip-amenable mutations include, but are not limited to, a deletion of exons 1-43, 2-43, 3-43, 4-43, 5-43, 6-43, 7-43, 8-43, 9-43, 10-43, 11-43, 12-43, 13-43, 14-43, 15-43, 16-43, 17-43, 18-43, 19-43, 20-43, 21-43, 22-43, 23-43, 24-43, 25-43, 26-43, 27-43, 28-43, 29-43, 30-43, 31-43, 32-43, 33-43, 34-43, 35-43, 36-43, 37-43, 38-43, 39-43, 40-43, 41-43, 42-43, 43, 45, 45-46, 45-47, 45-48, 45-49, 45-50, 45-51, 45-52, 45-53, 45-54, 45-55, 45-56, 45-57, 45-58, 45-59, 45-60, 45-61, 45-62, 45-63, 45-64, 45-65, 45-66, 45-67, 45-68, 45-69, 45-70, 45-71, 45-72, 45-73, 45-74, 45-75, 45-76, 45-77, and 45-78, and a duplication of exon 44. In some aspects, such mutations are a duplication of DMD exon 44, a deletion of exon 43 or 45, or a deletion of exons 45-56. The disclosure also provides vectors for delivering the nucleic acids described herein to a subject in need thereof.

The disclosure provides methods for delivering a nucleic acid (or nucleic acid molecule) comprising an antisense sequence or the reverse complement of the antisense sequence designed to target exon 44 or the intronic region surrounding exon 44. The disclosure provides methods for delivering a nucleic acid molecule encoding a U7 snRNA comprising an exon 44 targeting antisense sequence, an “exon 44-targeted U7snRNA polynucleotide construct.” In some aspects, the polynucleotide construct is inserted in the genome of a viral vector for delivery. In some aspects the vector used to deliver the exon 44-targeted U7snRNA polynucleotide construct is an rAAV.

The disclosure thus provides an rAAV to deliver a U7 small RNA promoter that will express the antisense of interest, thus mediating exon skipping. The advantage of this approach is that rAAV virus will efficiently target the affected muscle, where it will deliver the exon skipping system.

The DMD gene is the largest known gene in humans. It is 2.4 million base-pairs in size, comprises 79 exons and takes over 16 hours to be transcribed and cotranscriptionally spliced. In some aspects, the disclosure is directed to nucleic acid molecules comprising polynucleotide sequences targeting exon 44 of the DMD gene and vectors comprising such nucleic acid molecules to induce exon 44 skipping. The rationale of antisense-mediated exon skipping is to induce the skipping of a target exon to restore the reading frame. The polynucleotide sequence of exon 44 of the DMD gene with its surrounding intronic sequence is set out in SEQ ID NO: 1. The nucleotides in upper case indicate exonic sequence and the nucleotides in lower case indicate intronic sequence. The polynucleotide sequence of exon 44 of the DMD gene is set out in SEQ ID NO: 2 and consists of 148 base pairs (U.S. Patent Publication No. 2012/0059042), and the amino acid sequence of exon 44 is set out in SEQ ID NO: 3. The first “G” of SEQ ID NO: 2 is the terminal nucleotide encoding the final C-terminal amino acid in exon 43. Thus, although “G” is the first nucleotide in SEQ ID NO: 2, exon 44 starts to be coded by “CGA,” which encodes the N-terminal “R” (arginine) in SEQ ID NO: 3.

The disclosure provides a nucleic acid (or a nucleic acid molecule) or nucleic acids comprising or consisting of an antisense nucleotide sequence designed to target exon 44 of the DMD gene. Exon 44 of the DMD gene with surrounding intronic sequence comprises the nucleotide sequence set out in SEQ ID NO: 1. Exon 44 of the DMD gene comprises the nucleotide sequence set out in SEQ ID NO: 2 or encodes the amino acid sequence set out in SEQ ID NO: 3.

In various aspects, the methods of the disclosure also target isoforms and variants of the nucleotide sequence set forth in SEQ ID NO: 1 or 2, or the nucleotide sequence encoding the amino acid sequence set out in SEQ ID NO: 3. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 1 or 2 or the nucleotide sequence encoding the amino acid sequence set out in SEQ ID NO: 3. Table 1 provides the sequences of human DMD exon 44 and it surrounding intronic region.

TABLE 1
Human DMD Exon 44 - Polynucleotide and Amino Acid Sequences.

| Name and | SEQ | Sequence |
|---|---|---|
| description of | ID | |
| sequence | NO: | |
| hDMD - Exon | 1 | ttgtcagtataaccaaaaaatatacgctatatctctataatctgttttaca |
| 44 (upper | | taatccatctatttttcttgatccatatgcttttacctgcagGCGATTTGA |
| case) with | | CAGATCTGTTGAGAAATGGCGGCGTTTTCATTATGATATAAAGATATTTAA |
| surrounding | | TCAGTGGCTAACAGAAGCTGAACAGTTTCTCAGAAAGACACAAATTCCTGA |
| intronic | | GAATTGGGAACATGCTAAATACAAATGGTATCTTAAGgtaagtctttgatt |
| sequence | | tgttttttcgaaattgtatttatcttcagcacatctggactcttt |
| (lower case) | | |
| hDMD - Exon | 2 | GCGATTTGACAGATCTGTTGAGAAATGGCGGCGTTTTCATTATG |
| 44 nucleotide | | ATATAAAGATATTTAATCAGTGGCTAACAGAAGCTGAACAGTTT |
| sequence | | CTCAGAAAGACACAAATTCCTGAGAATTGGGAACATGCTAAATA |
| | | CAAATGGTATCTTAAG |
| hDMD - Exon | 3 | RFDRSVEKWRRFHYDIKIFNQWLTEAEQFLRKTQIPENWEHAKYK |
| 44 | | WYLK |
| amino acid | | |
| sequence | | |

The disclosure includes various nucleic acid molecules comprising target sequences of various regions in and around exon 44, including the sense and antisense sequences set out in Table 2, and their use in a method for inducing skipping of exon 44 of the DMD gene in a cell. Thus, the disclosure includes methods and uses for inducing skipping of exon 44 of the DMD gene in a cell comprising providing the cell with a nucleic acid molecule targeting exon 44, i.e., an “exon 44-targeted U7snRNA polynucleotide construct.” The disclosure therefore provides a nucleic acid molecule comprising antisense sequences targeting various regions of exon 44 and reverse complements of these sequences. The target sequences, i.e., native sequences of exon 44 that are being targeted by the antisense sequences include, but are not limited to, the sequences set forth in SEQ ID NO: 4 [BP43AS44 (branch point 43 acceptor site 44) target sequence], SEQ ID NO: 5 [LESE44 (long exon splicing enhancer 44) target sequence], SEQ ID NO: 6 [SESE44 (short exon splicing enhancer 44) target sequence], or SEQ ID NO: 7 [SD44 (splice donor) target sequence], or variants thereof. In some aspects, these target sequences are inserted into the U7-encoding sequences, i.e., SEQ ID NO: 29. In some aspects, these antisense sequences are inserted into the U7-encoding sequences, i.e., SEQ ID NO: 28. In some aspects, multiple copies of these sequences are inserted into the U7-encoding sequences. The disclosure also provides a nucleic acid molecule comprising sequences targeting various regions of exon 44, reverse complements of the target sequences, and mRNA sequences set forth in SEQ ID NO: 32 [mRNA of BP43AS44 target sequence], SEQ ID NO: 33 [mRNA of LESE44 target sequence], SEQ ID NO: 34 [mRNA of SESE44 target sequence], or SEQ ID NO: 35 [mRNA of SD44 target sequence], or variants thereof. See Table 2. The upper case letters in the sequences represent exonic sequence (i.e., sequence in exon 44) and the lower case letters in the sequences represent intronic sequence surrounding exon 44. These sequences are present in the DMD gene found within SEQ ID NO: 1 or 2.

TABLE 2
Target Sequences and Corresponding mRNA Sequences
in and Adjacent to Exon 44 of Human DMD.

| Name and | SEQ | |
|---|---|---|
| description | ID | |
| of sequence | NO: | Sequence |
| BP43AS44 | 4 | tttcttgatccatatgcttttacctgcagGCGATT |
| (Exonic | | TGACAGAT |
| sequence is | | |
| upper case; | | |
| surrounding | | |
| intronic | | |
| sequence is | | |
| lower case) | | |
| LESE44 | 5 | TCAGTGGCTAACAGAAGCTGAACAGTTTCTCAGAA |
| | | AGACACAA |
| SESE44 | 6 | TCAGTGGCTAACAGAAGCTGAACAGTTTCTCAGAA |
| SD44 | 7 | CAAATGGTATCTTAAGgtaag |
| (Exonic | | |
| sequence is | | |
| upper case; | | |
| surrounding | | |
| intronic | | |
| sequence is | | |
| lower case) | | |
| BP43AS44 | 32 | uuucuugauccauaugcuuuuaccugcagGCGAUU |
| mRNA | | UGACAGAU |
| LESE44 | 33 | UCAGUGGCUAACAGAAGCUGAACAGUUUCUCAGAA |
| mRNA | | AGACACAA |
| SESE44 | 34 | UCAGUGGCUAACAGAAGCUGAACAGUUUCUCAGAA |
| mRNA | | |
| SD44 mRNA | 35 | CAAAUGGUAUCUUAAGguaag |

The disclosure includes nucleic acid molecules comprising or consisting of antisense sequences (and sequences that are the reverse complement of the antisense sequences) that interfere with the expression of exon 44 of the DMD gene by interfering with the spliceosome resulting in the skipping of exon 44 of the DMD gene in order to restore the reading frame of the mRNA leading to expression of a truncated dystrophin protein in order to treat, ameliorate and/or prevent a muscular dystrophy resulting from a mutation in the DMD gene and the resultant altered version of mRNA. Thus, as used herein, “increased expression of dystrophin” includes “increased expression of a truncated dystrophin protein, an altered form or dystrophin protein, or a functional fragment of the dystrophin protein.” In some aspects, the disclosure includes antisense sequences that target exon 44 and its surrounding intronic sequence. In some aspects, the antisense sequences include the sequences set out in any of SEQ ID NOs: 8-11, or variant sequences thereof. In some aspects, the disclosure includes antisense mRNA sequences that target exon 44 and its surrounding intronic sequence. In some aspects, the mRNA sequences of these antisense sequences include the sequences set out in SEQ ID NOs: 12-15, or variants thereof. See Table 3. In some aspects, these antisense sequences or their reverse complements are inserted into the U7-encoding sequences, e.g., SEQ ID NO: 28 or 29. In some aspects, multiple copies of these sequences are inserted into the U7-encoding sequences.

TABLE 3
Antisense Sequences (Reverse Complementary
Sequences of the Target Sequence) and
Corresponding mRNA Sequences Binding to Exon
44 and Surrounding Intronic Sequence of Human
DMD.

| Name and | SEQ | |
|---|---|---|
| description | ID | |
| of sequence | NO: | Sequence |
| BP43AS44 | 8 | ATCTGICAAATCGCctgcaggtaaaagcatat |
| antisense | | ggatcaagaaa |
| (Exonic | | |
| sequence is | | |
| upper case; | | |
| surrounding | | |
| intronic | | |
| sequence is | | |
| lower case) | | |
| LESE44 | 9 | TTGTGTCTTTCTGAGAAACTGTTCAGCTTCTGT |
| antisense | | TAGCCACTGA |
| SESE44 | 10 | TTCTGAGAAACTGTTCAGCTTCTGTTAGCCACT |
| antisense | | GA |
| SD44 | 11 | cttacCTTAAGATACCATTTG |
| antisense | | |
| (Exonic | | |
| sequence is | | |
| upper case; | | |
| surrounding | | |
| intronic | | |
| sequence is | | |
| lower case) | | |
| BP43AS44 | 12 | AUCUGUCAAAUCGCcugcagguaaaagcauaug |
| antisense | | gaucaagaaa |
| mRNA | | |
| LESE44 | 13 | UUGUGUCUUUCUGAGAAACUGUUCAGCUUCUGU |
| antisense | | UAGCCACUGA |
| mRNA | | |
| SESE44 | 14 | UUCUGAGAAACUGUUCAGCUUCUGUUAGCCACU |
| antisense | | GA |
| mRNA | | |
| SD44 | 15 | cuuacCUUAAGAUACCAUUUG |
| antisense | | |
| mRNA | | |

The disclosure includes nucleic acids comprising any one or more of the sequences set forth in any of SEQ ID NOs: 4-15 or 32-35 under the control of a U7 promoter or inserted into a sequence encoding U7 small nuclear RNA (U7 snRNA). Such sequences encoding U7 snRNA are set out in SEQ ID NOs: 28 and 29 and can be found in Table 5. U7 snRNA have been found to be important tools in exon skipping and splicing modulation [Goyenvalle et al., Mol Ther 17(7):1234-40 (2009)]. Moreover, splicing modulation using antisense oligonucleotides (AONs) has been developed for the past two decades as a potential treatment for many diseases, most notably Duchene muscular dystrophy (DMD). This includes pre-clinical and clinical trials [Mendell et al., Ann Neurol 74:637-47 (2013)]. However, such AONs were only shown to mediate weak exon skipping due to the fact that they penetrate the heart and diaphragm (i.e., the most affected muscles in DMD boys) only weakly and they are not stable, i.e., requiring reinjection of DMD patients. It is therefore described herein that AAV-based U7 snRNA gene therapy approaches help circumvent the aforementioned potential delivery problems of AONs.

The disclosure includes nucleic acid molecules comprising or consisting of the nucleotide sequences encoding U7 snRNA (U7 snRNA antisense sequences, i.e., SEQ ID NOs: 16-19, 24, and 25, and reverse complement U7 snRNA antisense sequences, i.e., SEQ ID NOs: 20-23, 26, and 27), that interfere with the expression of exon 44 of the DMD gene by interfering with the spliceosome resulting in the skipping of exon 44 of the DMD gene in order to restore the reading frame of the mRNA leading to expression of a truncated dystrophin protein in order to treat, ameliorate and/or prevent a muscular dystrophy resulting from a mutation in the DMD gene and the resultant altered version of mRNA. See Table 4.

TABLE 4
Sequences Encoding U7 snRNA Sense and Antisense Sequences that Bind
Exon 44 and Surrounding Intronic Sequence of Human DMD.

| Name and | SEQ | |
|---|---|---|
| description of | ID | |
| sequence | NO: | Sequence |
| U7- | 16 | taacaacataggagctgtgattggctgttttcagccaatcagcactgactcatttgcatagccttt |
| BP43AS44 | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaaa |
| | | tctgtcaaatcgcctgcaggtaaaagcatatggatcaagaaaaatttttggagcaggttttctga |
| | | cttcggtcggaaaacccctcccaatttcactggtctacaatgaaagcaaaacagttctcttccc |
| | | cgctccccggtgtgtgagaggggctttgatccttctctggtttcctaggaaacgcgtatgtg |
| U7-LESE44 | 17 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaatt |
| | | gtgtctttctgagaaactgttcagcttctgttagccactgaaatttttggagcaggttttctgacttcg |
| | | gtcggaaaacccctcccaatttcactggtctacaatgaaagcaaaacagttctcttccccgctc |
| | | cccggtgtgtgagaggggctttgatccttctctggtttcctaggaaacgcgtatgtg |
| U7-SESE44 | 18 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaatt |
| | | ctgagaaactgttcagcttctgttagccactgaaatttttggagcaggttttctgacttcggtcgga |
| | | aaacccctcccaatttcactggtctacaatgaaagcaaaacagttctcttccccgctccccggt |
| | | gtgtgagaggggctttgatccttctctggtttcctaggaaacgcgtatgtg |
| U7-SD44 | 19 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaac |
| | | ttaccttaagataccatttgaatttttggagcaggttttctgacttcggtcggaaaacccctcccaa |
| | | tttcactggtctacaatgaaagcaaaacagttctcttccccgctccccggtgtgtgagaggggc |
| | | tttgatccttctctggtttcctaggaaacgcgtatgtg |
| U7-4xSD44 | 24 | taacaacataggagctgtgattggctgttttcagccaatcagcactgactcatttgcatagccttt |
| (comprises 4 | | acaagcggtcacaaactcaagaaacgagcggttttaatagtcttttagaatattgtttatcgaac |
| copies of the | | cgaataaggaactgtgctttgtgattcacatatcagtggaggggtgtggaaatggcaccttgat |
| SD44 insert) | | ctcaccctcatcgaaagtggagttgatgtccttccctggctcgctacagacgcacttccgcaac |
| | | ttaccttaagataccatttgaatttttggagcaggttttctgacttcggtcggaaaacccctcccaa |
| | | tttcactggtctacaatgaaagcaaaacagttctcttccccgctccccggtgtgtgagaggggc |
| | | tttgatccttctctggtttcctaggaaacgcgtatgtggctagataacaacataggagctgtgatt |
| | | ggctgttttcagccaatcagcactgactcatttgcatagcctttacaagcggtcacaaactcaa |
| | | gaaacgagcggttttaatagtcttttagaatattgtttatcgaaccgaataaggaactgtgctttgt |
| | | gattcacatatcagtggaggggtgtggaaatggcaccttgatctcaccctcatcgaaagtgga |
| | | gttgatgtccttccctggctcgctacagacgcacttccgcaacttaccttaagataccatttgaatt |
| | | tttggagcaggttttctgacttcggtcggaaaacccctcccaatttcactggtctacaatgaaag |
| | | caaaacagttctcttccccgctccccggtgtgtgagaggggctttgatccttctctggtttcctagg |
| | | aaacgcgtatgtggctagataacaacataggagctgtgattggctgttttcagccaatcagca |
| | | ctgactcatttgcatagcctttacaagcggtcacaaactcaagaaacgagcggttttaatagtct |
| | | tttagaatattgtttatcgaaccgaataaggaactgtgctttgtgattcacatatcagtggagggg |
| | | tgtggaaatggcaccttgatctcaccctcatcgaaagtggagttgatgtccttccctggctcgct |
| | | acagacgcacttccgcaacttaccttaagataccatttgaatttttggagcaggttttctgacttcg |
| | | gtcggaaaacccctcccaatttcactggtctacaatgaaagcaaaacagttctcttccccgctc |
| | | cccggtgtgtgagaggggctttgatccttctctggtttcctaggaaacgcgtatgtggctagata |
| | | acaacataggagctgtgattggctgttttcagccaatcagcactgactcatttgcatagcctttac |
| | | aagcggtcacaaactcaagaaacgagcggttttaatagtcttttagaatattgtttatcgaaccg |
| | | aataaggaactgtgctttgtgattcacatatcagtggaggggtgtggaaatggcaccttgatctc |
| | | accctcatcgaaagtggagttgatgtccttccctggctcgctacagacgcacttccgcaactta |
| | | ccttaagataccatttgaatttttggagcaggttttctgacttcggtcggaaaacccctcccaattt |
| | | cactggtctacaatgaaagcaaaacagttctcttccccgctccccggtgtgtgagaggggcttt |
| | | gatccttctctggtttcctaggaaacgcgtatgtg |
| U7-SD44- | 25 | ctagaggctcgagaagatatcaactgcagcttctactgggcggttttatggacagcaagcga |
| stuffer | | accggaattgccagctggggcgccctctggtaaggttgggaagccctgcaaagtaaactgg |
| (comprises | | atggctttctcgccgccaaggatctgatggcgcaggggatcaagctctgatcaagagacagg |
| SD44 with | | atgaggatcgtttcgcgttcttgactcttcgcgatgtacgggccagatatacgcgttgacattgat |
| stuffer | | tattgactagttattaatagtaatcaattacggggtcattagttcatagcccatatatggagttccg |
| sequence) | | cctgcagggacgtcgacggatcgggagatctcccgatcccctatctgctccctgcttgtgtgttg |
| | | gaggtcgctgagtagtgcgcgagcaaaatttaagctacaacaaggcaaggcttgaccgac |
| | | aattgcatgaagaatctgcttagggttaggcgttttgcgctgcttcgcggcgcgccttttaaggc |
| | | agttattggtgcccttaaacgcctggtgctacgcctgaataagtgataataagcggatgaatgg |
| | | cagaaattcgccggatctttgtgaaggaaccttacttctgtggtgtgacataattggacaaacta |
| | | cctacagagatttaaagctctactagggtgggcgaagaactccagcatgagatccccgcgct |
| | | ggaggatcatccagccggcgtcccggaaaacgattccgaagcccaacctttcatagaagg |
| | | cggcggtggaatcgaaatctcgtgatggcaggttgggcgtcgcttggtcggtcatttcgaaccc |
| | | cagagtcccgctcagggcgcgccgggggggggggcgctgaggtctgcctcgtgaagaag |
| | | gtgttgctgactcataccaggcctgaatcgccccatcatccagccagaaagtgagggagcc |
| | | acggttgatgagagctttgttgtaggtggaccagtcctgcaggagcataaagtgtaaagcctg |
| | | gggtgcctaatgagtgagctaactcacattaattgcgttgcgctcactgcccgctttccagtcgg |
| | | gaaacctgtcgtgcccgcccagtctagctatcgccatgtaagcccactgcaagctacctgcttt |
| | | ctctttgcgcttgcgttttcccttgtccagatagcccagtagctgacattcatccggggtcagcac |
| | | cgtttctgcggactggctttctacgtgtctggttcgaggcgggatcagccaccgcggtggcggc |
| | | ctagagtcgacgaggaactgaaaaaccagaaagttaactggcctgtacggaagtgttacttc |
| | | tgctctaaaagctgcggaattgtacccgcggccgatccaccggtcgccaccagcggccatc |
| | | aagcacgttatcgataccgtcgactagagctcgctgatcagtggggggtggggtggggcag |
| | | gacagcaagggggaggattgggaagacaatagcagctgcagaagtttaaacgcatgtaa |
| | | caacataggagctgtgattggctgttttcagccaatcagcactgactcatttgcatagcctttaca |
| | | agcggtcacaaactcaagaaacgagcggttttaatagtcttttagaatattgtttatcgaaccga |
| | | ataaggaactgtgctttgtgattcacatatcagtggaggggtgtggaaatggcaccttgatctca |
| | | ccctcatcgaaagtggagttgatgtccttccctggctcgctacagacgcacttccgcaacttac |
| | | cttaagataccatttgaatttttggagcaggttttctgacttcggtcggaaaacccctcccaatttc |
| | | actggtctacaatgaaagcaaaacagttctcttccccgctccccggtgtgtgagaggggctttg |
| | | atccttctctggtttcctaggaaacgcgtatgtg |
| Inverted or | 20 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttttcttgatccatatgcttttacctgcaggcgatttgacag |
| U7- | | atttgcggaagtgcgtctgtagcgagccagggaaggacatcaactccactttcgatgagggt |
| BP43AS44 | | gagatcaaggtgccatttccacacccctccactgatatgtgaatcacaaagcacagttccttat |
| | | tcggttcgataaacaatattctaaaagactattaaaaccgctcgtttcttgagtttgtgaccgcttg |
| | | taaaggctatgcaaatgagTcagtgctgattggctgaaaacagccaatcacagctcctatgtt |
| | | gtta |
| Inverted or | 21 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttcagtggctaacagaagctgaacagtttctcagaaag |
| U7-LESE44 | | acacaattgcggaagtgcgtctgtagcgagccagggaaggacatcaactccactttcgatga |
| | | gggtgagatcaaggtgccatttccacacccctccactgatatgtgaatcacaaagcacagttc |
| | | cttattcggttcgataaacaatattctaaaagactattaaaaccgctcgtttcttgagtttgtgacc |
| | | gcttgtaaaggctatgcaaatgagtcagtgctgattggctgaaaacagccaatcacagctcct |
| | | atgttgtta |
| Inverted or | 22 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttcagtggctaacagaagctgaacagtttctcagaattg |
| U7-SESE44 | | cggaagtgcgtctgtagcgagccagggaaggacatcaactccactttcgatgagggtgaga |
| | | tcaaggtgccatttccacacccctccactgatatgtgaatcacaaagcacagttccttattcggtt |
| | | cgataaacaatattctaaaagactattaaaaccgctcgtttcttgagtttgtgaccgcttgtaaag |
| | | gctatgcaaatgagtcagtgctgattggctgaaaacagccaatcacagctcctatgttgtta |
| Inverted or | 23 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| U7-SD44 | | agcgagccagggaaggacatcaactccactttcgatgagggtgagatcaaggtgccatttcc |
| | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgtta |
| Inverted or | 26 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| U7-4xSD44 | | agcgagccagggaaggacatcaactccactttcgatgagggtgagatcaaggtgccatttcc |
| | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgttatctagccacatacgcgtt |
| | | tcctaggaaaccagagaaggatcaaagcccctctcacacaccggggagcggggaagag |
| | | aactgttttgctttcattgtagaccagtgaaattgggaggggttttccgaccgaagtcagaaaac |
| | | ctgctccaaaaattcaaatggtatcttaaggtaagttgcggaagtgcgtctgtagcgagccag |
| | | ggaaggacatcaactccactttcgatgagggtgagatcaaggtgccatttccacacccctcc |
| | | actgatatgtgaatcacaaagcacagttccttattcggttcgataaacaatattctaaaagacta |
| | | ttaaaaccgctcgtttcttgagtttgtgaccgcttgtaaaggctatgcaaatgagtcagtgctgaft |
| | | ggctgaaaacagccaatcacagctcctatgttgttatctagccacatacgcgtttcctaggaaa |
| | | ccagagaaggatcaaagcccctctcacacaccggggagcggggaagagaactgttttgctt |
| | | tcattgtagaccagtgaaattgggaggggttttccgaccgaagtcagaaaacctgctccaaa |
| | | aattcaaatggtatcttaaggtaagttgcggaagtgcgtctgtagcgagccagggaaggaca |
| | | tcaactccactttcgatgagggtgagatcaaggtgccatttccacacccctccactgatatgtg |
| | | aatcacaaagcacagttccttattcggttcgataaacaatattctaaaagactattaaaaccgc |
| | | tcgtttcttgagtttgtgaccgcttgtaaaggctatgcaaatgagtcagtgctgattggctgaaaa |
| | | cagccaatcacagctcctatgttgttatctagccacatacgcgtttcctaggaaaccagagaa |
| | | ggatcaaagcccctctcacacaccggggagcggggaagagaactgttttgctttcattgtaga |
| | | ccagtgaaattgggaggggttttccgaccgaagtcagaaaacctgctccaaaaattcaaatg |
| | | gtatcttaaggtaagttgcggaagtgcgtctgtagcgagccagggaaggacatcaactccac |
| | | tttcgatgagggtgagatcaaggtgccatttccacacccctccactgatatgtgaatcacaaag |
| | | cacagttccttattcggttcgataaacaatattctaaaagactattaaaaccgctcgtttcttgagt |
| | | ttgtgaccgcttgtaaaggctatgcaaatgagtcagtgctgattggctgaaaacagccaatca |
| | | cagctcctatgttgtta |
| Inverted or | 27 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| SD44-stuffer | | agcgagccagggaaggacatcaactccactttcgatgagggtgagatcaaggtgccatttcc |
| | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgttacatgcgtttaaacttctgc |
| | | agctgctattgtcttcccaatcctcccccttgctgtcctgccccaccccaccccccactgatcag |
| | | cgagctctagtcgacggtatcgataacgtgcttgatggccgctggtggcgaccggtggatcgg |
| | | ccgcgggtacaattccgcagcttttagagcagaagtaacacttccgtacaggccagttaacttt |
| | | ctggtttttcagttcctcgtcgactctaggccgccaccgcggtggctgatcccgcctcgaacca |
| | | gacacgtagaaagccagtccgcagaaacggtgctgaccccggatgaatgtcagctactgg |
| | | gctatctggacaagggaaaacgcaagcgcaaagagaaagcaggtagcttgcagtgggctt |
| | | acatggcgatagctagactgggcgggcacgacaggtttcccgactggaaagcgggcagtg |
| | | agcgcaacgcaattaatgtgagttagctcactcattaggcaccccaggctttacactttatgctc |
| | | ctgcaggactggtccacctacaacaaagctctcatcaaccgtggctccctcactttctggctgg |
| | | atgatggggcgattcaggcctggtatgagtcagcaacaccttcttcacgaggcagacctcag |
| | | cgcccccccccccggcgcgccctgagcgggactctggggttcgaaatgaccgaccaagcg |
| | | acgcccaacctgccatcacgagatttcgattccaccgccgccttctatgaaaggttgggcttcg |
| | | gaatcgttttccgggacgccggctggatgatcctccagcgcggggatctcatgctggagttctt |
| | | cgcccaccctagtagagctttaaatctctgtaggtagtttgtccaattatgtcacaccacagaag |
| | | taaggttccttcacaaagatccggcgaatttctgccattcatccgcttattatcacttattcaggcg |
| | | tagcaccaggcgtttaagggcaccaataactgccttaaaaggcgcgccgcgaagcagcgc |
| | | aaaacgcctaaccctaagcagattcttcatgcaattgtcggtcaagccttgccttgttgtagctta |
| | | aattttgctcgcgcactactcagcgacctccaacacacaagcagggagcagataggggatc |
| | | gggagatctcccgatccgtcgacgtccctgcaggcggaactccatatatgggctatgaacta |
| | | atgaccccgtaattgattactattaataactagtcaataatcaatgtcaacgcgtatatctggccc |
| | | gtacatcgcgaagagtcaagaacgcgaaacgatcctcatcctgtctcttgatcagagcttgat |
| | | cccctgcgccatcagatccttggcggcgagaaagccatccagtttactttgcagggcttccca |
| | | accttaccagagggcgccccagctggcaattccggttcgcttgctgtccataaaaccgccca |
| | | gtagaagctgcagttgatatcttctcgagcctctag |

The disclosure therefore includes nucleic acids (i.e., nucleic acid molecules or nucleic acid constructs) comprising one or more of the nucleotide sequences set out in any of SEQ ID NOs: 4-27 and 32-35, or comprising one or more of nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the nucleotide sequence set out in any of SEQ ID NOs: 4-27 and 32-35.

In some aspects, the disclosure uses U7 snRNA molecules comprising the nucleotide sequences described herein to inhibit or interfere with splicing. U7 snRNA is normally involved in histone pre-mRNA 3′ end processing but, in some aspects, it is converted into a versatile tool for splicing modulation or as antisense RNA that is continuously expressed in cells [Goyenvalle et al., Science 306(5702): 1796-9 (2004)]. By replacing the wild-type U7 Sm binding site with a consensus sequence derived from spliceosomal snRNAs, the resulting RNA assembles with the seven Sm proteins found in spliceosomal snRNAs. As a result, this U7 Sm OPT RNA accumulates more efficiently in the nucleoplasm and no longer mediates histone pre-mRNA cleavage, although it can still bind to histone pre-mRNA and act as a competitive inhibitor for wild-type U7 small nuclear ribonucleoproteins (snRNPs). By further replacing the sequence binding to the histone downstream element with one complementary to a particular target in a splicing substrate, it is possible to create U7 snRNAs capable of modulating specific splicing events. One advantage of using U7 derivatives is that the antisense sequence is embedded into a small nuclear ribonucleoprotein (snRNP) complex. Moreover, when embedded into a gene therapy vector, these small RNAs can be permanently expressed inside the target cell after a single injection and their use using an AAV approach has been investigated in vivo [Levy et al., Eur J Hum Genet 18(9): 969-70 (2010); Wein et al., Hum Mutat 31(2): 136-42 (2010); Wein et al., Nat Med 20(9): 992-1000 (2014)].

There are three major features to the U7-snRNA system: the U7 promoter to drive expression of (1) the modified snRNA in target cells; (2) an antisense sequence inserted in the snRNA backbone, which is designed to base-pair with splice junctions, branch points, or splicing enhancers; (3) a modified sequence (called smOPT) which recruits a distinct ring of RNA binding proteins that complexes with the U7snRNA making it more stable. [Schumperli et al., Cell and Mol Life Sciences 61:2560-70 (2004)]. It is noteworthy that the antisense sequence and the U7 small nuclear RNA (snRNA) (U7 snRNA) have proven safe for use in vivo in large animal models of muscular dystrophy [LeGuiner et al., Mol Ther 22:1923-35 (2014)].

The disclosure includes nucleic acid molecules comprising or consisting of a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the nucleotide sequence set out in any of SEQ ID NOs: 4-27 and 32-35.

Thus, the disclosure provides nucleic acids, including nucleic acids encoding target sequence, nucleic acids encoding antisense sequences and reverse complements of the antisense sequences, nucleic acids encoding U7-based small nuclear ribonucleic acids (snRNAs), i.e., U7-based snRNAs, nucleic acids encoding the reverse complement of the U7-based snRNAs, and recombinant adeno-associated virus (rAAV) comprising the nucleic acid molecules to deliver nucleic acids encoding U7-based snRNAs to induce exon-skipping for use in treating a muscular dystrophy.

In some aspects, the disclosure includes complete constructs (referred to herein as exon 44 U7 snRNA polynucleotide constructs, or exon 44-targeted U7 snRNA), which inhibit or interfere with the expression and/or incorporation of exon 44 of the DMD gene into the mRNA. Thus, the disclosure provides nucleic acid sequences encoding (1) exon 44-targeted U7snRNA-encoding polynucleotides (e.g., SEQ ID NOs: 16-19, 24, and 25), and (2) exon 44-targeted reverse complementary U7 snRNA-encoding polynucleotides (e.g., SEQ ID NOs: 20-23, 26, and 27).

Thus, the disclosure includes nucleic acids comprising or consisting of a nucleotide sequence that binds to any of the target sequences set forth in SEQ ID NOs: 1-7, nucleic acids comprising or consisting of a nucleotide sequence that is an antisense sequence (reverse complement of the targeted sequence at the DNA level) designed to target exon 44 and its surrounding intronic sequence (i.e., SEQ ID NOs: 8-11), nucleic acids comprising or consisting of a nucleotide sequence that is a reverse complementary sequence (reverse complement of the targeted sequence at the RNA level) designed to target exon 44 and its surrounding intronic sequence (i.e., SEQ ID NOs: 12-15), nucleic acids that encode U7 snRNA comprising or consisting of at least one or more of the nucleotide sequences set forth in SEQ ID NOs: 4-15 and 32-35, and nucleic acids comprising or consisting of at least one or more of the nucleotide sequences set forth in SEQ ID NOs: 16-27. The disclosure contemplates that the nucleic acids encoding these inhibitory splicing RNAs are responsible for sequence-specific gene exon skipping. In some aspects, the herein described nucleic acids or nucleic acid molecules or constructs are inserted into a vector.

Thus, the disclosure includes vectors comprising the nucleic acids described herein. In some aspects, more than one of any of these nucleic acids are combined into a single vector. Thus, in some aspects, combinations of exon 44-targeted nucleic acids or exon 44-targeted U7 snRNA constructs are present in a single vector. The disclosure therefore includes vectors comprising one or more of the nucleotide sequences set out in SEQ ID NOs: 4-27 and 32-35 or nucleotide sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the nucleotide sequence set out in any of SEQ ID NOs: 4-27 and 32-35. In some aspects, the vectors are viral vectors, such as adeno-associated virus (AAV), adenovirus, retrovirus, lentivirus, equine-associated virus, alphavirus, pox viruses, herpes virus, polio virus, sindbis virus and vaccinia viruses) to deliver polynucleotides encoding antisense sequences mediating DMD exon 44 skipping as disclosed herein. In some aspects, adeno-associated virus (AAV) is used. In some aspects, recombinant adeno-associated virus (rAAV) is used.

In some aspects, rAAV genomes of the disclosure comprise one or more AAV ITRs flanking a polynucleotide encoding, for example, one or more DMD exon 44 U7-based snRNAs (i.e., an snRNA that binds to a gene sequence within or surrounding exon 44 and is expressed from a U7 snRNA). The polynucleotide is operatively linked to transcriptional control DNA, specifically promoter DNA that is functional in target cells.

Adeno-associated virus (AAV) is a replication-deficient parvovirus, the single-stranded DNA genome of which is about 4.7 kb in length including two 145 nucleotide inverted terminal repeat (ITRs) and the double-stranded DNA genome of which is about 2.3 kb in length, including two 145 nucleotide ITRs. There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J Virol, 45: 555-64 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAVrh74 genome; the AAV-9 genome is provided in Gao et al., J Virol, 78: 6381-8 (2004); the AAV-10 genome is provided in Mol Ther 13(1): 67-76 (2006); the AAV-11 genome is provided in Virology, 330(2): 375-83 (2004); the genome of AAV-12 is provided in GenBank Accession No. DQ813647.1; and the genome of AAV-13 is provided in GenBank Accession No. EU285562.1. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV genomes of the disclosure comprise one or more AAV ITRs flanking at least one exon 44-targeted U7 snRNA polynucleotide construct. Genomes with exon 44-targeted U7 snRNA polynucleotide constructs comprising each of the exon 44 targeting antisense sequences as described herein are specifically contemplated, as well as genomes with exon 44-targeted U7 snRNA polynucleotide constructs comprising each possible combination of two or more of the exon 44 targeting antisense sequences described herein. In some embodiments, including the exemplified embodiments, the U7 snRNA polynucleotide includes its own promoter.

AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12 and AAV-13, AAV-rh74, and AAV-anc80. The nucleotide sequences of the genomes of these various AAV serotypes are known in the art. In some embodiments of the disclosure, the promoter DNAs are muscle-specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol. Cell. Biol., 11: 4854-4862 (1991)], control elements derived from the human skeletal actin gene [Muscat et al., Mol. Cell. Biol., 7: 4089-4099 (1987)], the cardiac actin gene, muscle creatine kinase sequence elements [Johnson et al., Mol. Cell. Biol., 9:3393-3399 (1989)] and the murine creatine kinase enhancer (MCK) element, desmin promoter, control elements derived from the skeletal fast-twitch troponin C gene, the slow-twitch cardiac troponin C gene and the slow-twitch troponin I gene: hypozia-inducible nuclear factors [Semenza et al., Proc. Natl. Acad. Sci. USA, 88: 5680-5684 (1991)], steroid-inducible elements and promoters including the glucocorticoid response element (GRE) [See Mader and White, Proc. Natl. Acad. Sci. USA, 90: 5603-5607 (1993)], and other control elements.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12 and AAV-13, AAV-rh74, and AAV-anc80. Use of cognate components is specifically contemplated. Production of pseudotyped rAAV is disclosed in, for example, WO 01/83692 which is incorporated by reference herein in its entirety.

In some embodiments of the disclosure, the virus genome is a single-stranded genome or a self-complementary genome. In some embodiments of the methods, the genome of the rAAV lacks AAV rep and cap DNA.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., Proc Natl Acad Sci USA, 79:2077-81 (1982)], addition of synthetic linkers containing restriction endonuclease cleavage sites [Laughlin et al., Gene, 23:65-73 (1983)] or by direct, blunt-end ligation [Senapathy et al., J Biol Chem 259:4661-6 (1984)]. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology, 1533-539 (1992); and Muzyczka, Curr Topics in Microbial and Immunol, 158:97-129 (1992)). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988); Samulski et al., J. Virol., 63:3822-8 (1989); U.S. Pat. No. 5,173,414; WO 95/13365 and corresponding U.S. Pat. No. 5,658,776; WO 95/13392; WO 96/17947; PCT/U598/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-50 (1995); Paul et al., Human Gene Therapy 4:609-615 (1993); Clark et al., Gene Therapy 3:1124-32 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Cell transduction efficiencies of the methods of the disclosure described above and below may be at least about 60, 65, 70, 75, 80, 85, 90 or 95 percent efficient.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther. 10(6): 1031-9 (1999); Schenpp et al., Methods Mol. Med. 69:427-43 (2002); U.S. Pat. No. 6,566,118; and WO 98/09657.

In another embodiment, the disclosure contemplates compositions comprising rAAV comprising any of the nucleic acid molecules or constructs described herein. In one aspect, the disclosure includes a composition comprising the rAAV for delivering the snRNAs described herein. Compositions of the disclosure comprise rAAV in a pharmaceutically acceptable carrier. The compositions may also comprise other ingredients such as diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, respectively).

In some aspects, the disclosure provides a method of delivering DNA encoding the snRNA set out in any of SEQ ID NO: 4-27 and 32-35 to a subject in need thereof, comprising administering to the subject an rAAV encoding the exon 44-targeted snRNA. In some aspects, the disclosure provides AAV transducing cells for the delivery of the exon 44-targeted snRNAs.

Methods of transducing a target cell (e.g., a skeletal muscle) with rAAV, in vivo or in vitro, are contemplated by the disclosure. The methods comprise the step of administering an effective dose, or effective multiple doses, of a composition comprising a rAAV of the disclosure to an animal (including a human being) in need thereof. If the dose is administered prior to development of a muscular dystrophy, e.g., DMD, the administration is prophylactic. If the dose is administered after the development of a muscular dystrophy, the administration is therapeutic. In embodiments of the disclosure, an effective dose is a dose that alleviates (eliminates or reduces) at least one symptom associated with a muscular dystrophy being treated, that slows or prevents progression of the muscular dystrophy, e.g. DMD, that slows or prevents progression of the muscular dystrophy disorder/disease state, that diminishes the extent of disease, that results in remission (partial or total) of disease, and/or that prolongs survival of the subject suffering from the disorder or disease.

Administration of an effective dose of the compositions may be by routes standard in the art including, but not limited to, intramuscular, parenteral, intravenous, intrathecal, oral, buccal, nasal, pulmonary, intracranial, intraosseous, intraocular, rectal, or vaginal. Route(s) of administration and serotype(s) of AAV components of rAAV (in particular, the AAV ITRs and capsid protein) of the disclosure may be chosen and/or matched by those skilled in the art taking into account the infection and/or disease state being treated and the target cells/tissue(s). In some embodiments, the route of administration is intramuscular. In some embodiments, the route of administration is intravenous.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) are specifically contemplated, as are combinations with other therapies such as those disclosed in International Publication No. WO 2013/016352, which is incorporated by reference herein in its entirety.

Administration of an effective dose of the compositions may be by routes standard in the art including, but not limited to, intramuscular, parenteral, intravenous, intrathecal, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the exon 44 targeted U7-based snRNAs.

In particular, actual administration of rAAV of the disclosure is, in some aspects, accomplished by using any physical method that will transport the rAAV vector into the target tissue of a subject. Administration according to the disclosure includes, but is not limited to, injection into muscle, the liver, the cerebral spinal fluid, or the bloodstream. Simply resuspending an rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). In some aspects, capsid proteins of an rAAV are modified so that the rAAV is targeted to a particular target tissue of interest, such as muscle. See, for example, WO 02/053703, the disclosure of which is incorporated by reference herein. In some aspects, compositions or pharmaceutical compositions are prepared as injectable formulations or as topical formulations to be delivered to the muscles by transdermal transport. Numerous formulations for both intramuscular injection and transdermal transport have been previously developed and can be used in the practice of the disclosure. In some aspects, the rAAV are used with any pharmaceutically acceptable carrier or excipient for ease of administration and handling.

In some aspects, for purposes of intramuscular injection, solutions in an adjuvant, such as sesame or peanut oil or in aqueous propylene glycol, are employed, as well as sterile aqueous solutions. Such aqueous solutions, in various aspects, are buffered, if desired, and the liquid diluent is rendered isotonic with saline or glucose. In some aspects, solutions of rAAV as a free acid (DNA contains acidic phosphate groups) or a pharmacologically acceptable salt are prepared in water, suitably mixed with a surfactant such as hydroxpropylcellulose. In various aspects, a dispersion of rAAV is prepared in glycerol, liquid polyethylene glycol(s) and mixtures thereof and in oils. Under ordinary conditions of storage and use, these preparations contain a preservative to prevent the growth of microorganisms. In this connection, the sterile aqueous media employed are all readily obtainable by standard techniques in the art.

Formulations, including pharmaceutical forms suitable for injectable use, include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms, such as bacteria and fungi. In some aspects, the carrier is a solvent or dispersion medium containing, for example, water, ethanol, polyol (e.g., glycerol, propylene glycol, liquid polyethylene glycol, and the like), suitable mixtures thereof, and vegetable oils. Proper fluidity, in some aspects, is maintained by the use of a coating, such as lecithin, by the maintenance of the required particle size, in the case of a dispersion, and by the use of surfactants. The prevention of the action of microorganisms can be brought about by various antibacterial and antifungal agents, for example, parabens, chlorobutanol, phenol, sorbic acid, thimerosal, and the like. In some aspects, it is preferable to include isotonic agents, for example, sugars or sodium chloride. Prolonged absorption of the injectable compositions, in some aspects, is brought about by use of agents delaying absorption, for example, aluminum monostearate and gelatin.

Sterile injectable solutions are prepared, in some aspects, by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, various methods of preparation are vacuum drying and the freeze drying technique that yield a powder of the active ingredient plus any additional desired ingredient from the previously sterile-filtered solution thereof.

Transduction with rAAV, in some aspects, is also carried out in vitro. In one embodiment, for example, desired target muscle cells are removed from the subject, transduced with rAAV and reintroduced into the subject. Alternatively, syngeneic or xenogeneic muscle cells, in some aspects, are used where those cells will not generate an inappropriate immune response in the subject.

Suitable methods for the transduction and reintroduction of transduced cells into a subject are known in the art. In one embodiment, cells are transduced in vitro by combining rAAV with muscle cells, e.g., in appropriate media, and screening for those cells harboring the DNA of interest using conventional techniques in the art, such as Southern blots and/or PCR, or by using selectable markers. Transduced cells, in some aspects, are then formulated into a composition, including a pharmaceutical composition, and the composition is introduced into the subject by various techniques, such as by intramuscular, intravenous, subcutaneous, and/or intraperitoneal injection, or by injection into smooth and cardiac muscle, using e.g., a catheter.

The disclosure provides methods of administering an effective dose (or doses, administered essentially simultaneously or doses given at intervals) of rAAV that encode inhibitory RNAs and rAAV that encode combinations of inhibitory RNAs, including snRNAs, that target exon 44, and skipping of exon 44, to a subject in need thereof.

Transduction of cells with rAAV of the invention results in sustained expression of the exon 44 U7-based snRNAs. The term “transduction” is used to refer to the administration/delivery of one or more exon 44-targeted U7snRNA polynucleotide construct to a recipient cell either in vivo or in vitro, via a replication-deficient rAAV of the invention resulting in expression of the one or more exon 44-targeted U7snRNA polynucleotide construct by the recipient cell. The disclosure thus provides methods of administering/delivering rAAV which express exon 44 U7-based snRNAs to a subject. In some aspects, the subject is a human being.

These methods include transducing the blood and vascular system, the central nervous system, and tissues (including, but not limited to, tissues, such as muscle, organs such as liver and brain, and glands such as salivary glands) with one or more rAAV of the disclosure. Transduction, in some aspects, is carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et al., Science, 251: 761-6 (1991)], the myocyte-specific enhancer binding factor MEF-2 [Cserjesi et al., Mol Cell Biol 11: 4854-62 (1991)], control elements derived from the human skeletal actin gene [Muscat et al., Mol Cell Biol, 7: 4089-99 (1987)], the cardiac actin gene, muscle creatine kinase sequence elements [See Johnson et al., Mol Cell Biol, 9:3393-9 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors [Semenza et al., Proc Natl Acad Sci USA, 88: 5680-4 (1991)], steroid-inducible elements and promoters including the glucocorticoid response element (GRE) [See Mader et al., Proc Natl Acad Sci USA 90: 5603-7 (1993)], and other control elements.

Because AAV targets every dystrophin affected organ, the disclosure includes the delivery of DNAs encoding the inhibitory RNAs to all cells, tissues, and organs of a subject. In some aspects, the blood and vascular system, the central nervous system, muscle tissue, the heart, and the brain are attractive targets for in vivo DNA delivery. The disclosure includes the sustained expression of snRNA from transduced cells to affect DMD exon 44 expression (e.g., skip, knockdown or inhibit expression) and alter expression of the DMD protein. In some aspects, muscle tissue is targeted for delivery of the nucleic acid molecules and vectors of the disclosure. Muscle tissue is an attractive target for in vivo DNA delivery, because it is not a vital organ and is easy to access. The disclosure, in some aspects, contemplates sustained expression of one or more exon 44 U7-based snRNAs from transduced myofibers. By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells, in some aspects, are differentiated or undifferentiated, such as myoblasts, myocytes, myotubes, cardiomyocytes and cardiomyoblasts.

In yet another aspect, the disclosure provides a method of restoring the open reading frame of the DMD gene in a cell comprising contacting the cell with a rAAV encoding a exon 44-targeted U7 snRNA, wherein the RNA is encoded by the nucleotide sequence set out in at least one or more of any one of SEQ ID NOs: 4-27 and 32-35. In some aspects, skipping of exon 44 results in exclusion or inhibition of exon 44 by at least about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

Thus, the disclosure provides methods of administering an effective dose (or doses, administered essentially simultaneously or doses given at intervals) of an exon 44-targeted U7snRNA polynucleotide construct or an rAAV that comprises a genome that encodes one or more exon 44-targeted U7snRNA polynucleotide construct to a subject in need thereof (e.g., a subject or patient suffering from a muscular dystrophy, such as DMD).

In some aspects, a method of treating muscular dystrophy in a patient is provided. In some aspects, “treating” includes ameliorating, inhibiting, or even preventing one or more symptoms of a muscular dystrophy, including a duchenne muscular dystrophy, (including, but not limited to, muscle wasting, muscle weakness, skeletal muscle problems, heart function abnormalities, breathing difficulties, issues with speech and swallowing (dysarthria and dysphagia) or cognitive impairment). In some aspects, the method of treating results in increased expression of dystrophin protein or increased expression of an altered form or fragment of dystrophin protein that is physiologically or functionally active in the subject. In particular aspects, the method of treating inhibits the progression of dystrophic pathology in the subject. In some aspects, the method of treating improves muscle function in the subject. In some aspects, the improvement in muscle function is an improvement in muscle strength. In some aspects, the improvement in muscle function is an improvement in stability in standing and walking. The improvement in muscle strength is determined by techniques known in the art, such as the maximal voluntary isometric contraction testing (MVICT). In some instances, the improvement in muscle function is an improvement in stability in standing and walking. In some aspects, an improvement in stability or strength is determined by techniques known in the art such as the 6-minute walk test (6 MWT), the 100 meter run/walk test, or timed stair climb.

In some embodiments, the method of treating comprises the step of administering one or more exon 44 U7-based snRNA polynucleotide construct without the use of a vector. In some embodiments, the method of treating comprises the step of administering an rAAV to the subject, wherein the genome of the rAAV comprises one or more exon 44 U7-based snRNA polynucleotide construct.

In yet another aspect, the disclosure provides a method of inhibiting the progression of dystrophic pathology associated with a muscular dystrophy, such as DMD. In some embodiments, the method comprises the step of administering one or more exon 44 U7-based snRNA polynucleotide construct without the use of a vector. In some embodiments, the method comprises the step of administering an rAAV to the patient, wherein the genome of the rAAV comprises an exon 44-targeted U7snRNA polynucleotide construct.

Each publication, patent application, patent, and other reference cited herein is incorporated by reference in its entirety to the extent that it is not inconsistent with the present disclosure.

Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

All methods described herein are performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Design and Generation of Sequences that Target Exon 44

In order to test the ability of the U7snRNA system to induce skipping of exon 44, six AAV1-U7snRNAs were made. Antisense sequences (i.e., SEQ ID NOs: 8-27) were designed to bind “exon definition” (branchpoint, splice donor or acceptor, and exonic splicing enhancer) in order to exclude an exon (e.g., exon 44) from the mRNA. This “exon definition” can be predicted using the online software Human Splicing Finder (HSF, http colon-slash-slash-www.umd.be-slash-HSF-slash-HSF.shtml). The inventors used this software to design various target sequences and various targeting sequences with varying lengths and various binding sites. Sequences were commercially synthesized (GenScript).

The following table (i.e., Table 5 below) provides the sequences (nucleotide and amino acids) of exon 44 of the DMD gene (and intronic sequence surrounding exon 44), target sequences on the DMD gene (exon 44 sequence (in upper case letters in SEQ ID NO: 1) and intronic sequence surrounding exon 44 (in lower case letters in SEQ ID NO: 1)), antisense sequences used to target the sequences on the DMD gene (exon 44 and intronic sequence surrounding exon 44), reverse complement of the antisense sequences used to target the sequences on the DMD gene (exon 44 and intronic sequence surrounding exon 44), U7 sequences comprising antisense sequences used to target the sequences on the DMD gene (exon 44 and intronic sequence surrounding exon 44), and reverse complement of the U7 sequences comprising antisense sequences used to target the sequences on the DMD gene (exon 44 and intronic sequence surrounding exon 44).

Plasmids containing each of the constructs set out in SEQ ID NOs: 16-27 were amplified, resequenced and sent to the Viral Vector Core (VVC) at Nationwide Children's Hospital for insertion into a recombinant adeno-associated virus (rAAV) vector (i.e., between the ITRS). For the in vitro transduction studies, the constructs were produced using an AAV1 capsid. For in vivo studies, the constructs are produced into any AAV capsids as described herein.

TABLE 5
Sequences of the Disclosure.

| | SEQ | |
|---|---|---|
| Sequence | ID | |
| Name | NO: | Sequence |
| Human DMD | | |
| (hDMD) | | |
| hDMD - Exon | 1 | ttgtcagtataaccaaaaaatatacgctatatctctataatctgttttacataatccatctatttttctt |
| 44 (upper | | gatccatatgcttttacctgcagGCGATTTGACAGATCTGTTGAGAAATGG |
| case) with | | CGGCGTTTTCATTATGATATAAAGATATTTAATCAGTGGCTAAC |
| surrounding | | AGAAGCTGAACAGTTTCTCAGAAAGACACAAATTCCTGAGAATT |
| intronic | | GGGAACATGCTAAATACAAATGGTATCTTAAGgtaagtctttgatttgtttttt |
| sequence | | cgaaattgtatttatcttcagcacatctggactcttt |
| (lower case) | | |
| hDMD - Exon | 2 | GCGATTTGACAGATCTGTTGAGAAATGGCGGCGTTTTCATTATG |
| 44 nucleotide | | ATATAAAGATATTTAATCAGTGGCTAACAGAAGCTGAACAGTTT |
| sequence | | CTCAGAAAGACACAAATTCCTGAGAATTGGGAACATGCTAAATA |
| | | CAAATGGTATCTTAAG |
| hDMD - Exon | 3 | RFDRSVEKWRRFHYDIKIFNQWLTEAEQFLRKTQIPENWEHAKYK |
| 44 | | WYLK |
| amino acid | | |
| sequence | | |
| BP43AS44 | | |
| BP43AS44 | 4 | tttcttgatccatatgcttttacctgcagGCGATTTGACAGAT |
| target | | |
| sequence | | |
| 5′ part of Exon | | |
| 44 (upper | | |
| case) with | | |
| surrounding | | |
| intronic | | |
| sequence | | |
| (lower case) | | |
| BP43AS44 | 32 | uuucuugauccauaugcuuuuaccugcagGCGAUUUGACAGAU |
| target mRNA | | |
| sequence | | |
| BP43AS44 | 8 | ATCTGICAAATCGCctgcaggtaaaagcatatggatcaagaaa |
| antisense | | |
| sequence | | |
| BP43AS44 | 12 | AUCUGUCAAAUCGCcugcagguaaaagcauauggaucaagaaa |
| antisense | | |
| mRNA | | |
| sequence | | |
| U7- | 16 | taacaacataggagctgtgattggctgttttcagccaatcagcactgactcatttgcatagccttt |
| BP43AS44 | | acaagcggtcacaaactcaagaaacgagcggttttaatagtcttttagaatattgtttatcgaac |
| (also referred | | cgaataaggaactgtgctttgtgattcacatatcagtggaggggtgtggaaatggcaccttgat |
| to herein as | | ctcaccctcatcgaaagtggagttgatgtccttccctggctcgctacagacgcacttccgcaaa |
| pscAAV_shuttle_ | | tctgtcaaatcgcctgcaggtaaaagcatatggatcaagaaaaatttttggagcaggttttctga |
| KanR_bp4 | | cttcggtcggaaaacccctcccaatttcactggtctacaatgaaagcaaaacagttctcttccc |
| 3as44) | | cgctccccggtgtgtgagaggggctttgatccttctctggtttcctaggaaacgcgtatgtg |
| Inverted or | 20 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttttcttgatccatatgcttttacctgcaggcgatttgacag |
| U7- | | atttgcggaagtgcgtctgtagcgagccagggaaggacatcaactccactttcgatgagggt |
| BP43AS44 | | gagatcaaggtgccatttccacacccctccactgatatgtgaatcacaaagcacagttccttat |
| | | tcggttcgataaacaatattctaaaagactattaaaaccgctcgtttcttgagtttgtgaccgcttg |
| | | taaaggctatgcaaatgagtcagtgctgattggctgaaaacagccaatcacagctcctatgtt |
| | | gtta |
| LESE44 | | |
| LESE44 | 5 | TCAGTGGCTAACAGAAGCTGAACAGTTTCTCAGAAAGACACAA |
| target | | |
| sequence | | |
| LESE44 | 33 | UCAGUGGCUAACAGAAGCUGAACAGUUUCUCAGAAAGACACA |
| target mRNA | | A |
| sequence | | |
| LESE44 | 9 | TTGTGTCTTTCTGAGAAACTGTTCAGCTTCTGTTAGCCACTGA |
| antisense | | |
| sequence | | |
| LESE44 | 13 | UUGUGUCUUUCUGAGAAACUGUUCAGCUUCUGUUAGCCACU |
| antisense | | GA |
| mRNA | | |
| sequence | | |
| U7-LESE44 | 17 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaatt |
| | | gtgtctttctgagaaactgttcagcttctgttagccactgaaatttttggagcaggttttctgacttcg |
| | | gtcggaaaacccctcccaatttcactggtctacaatgaaagcaaaacagttctcttccccgctc |
| | | cccggtgtgtgagaggggctttgatccttctctggtttcctaggaaacgcgtatgtg |
| Inverted or | 21 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttcagtggctaacagaagctgaacagtttctcagaaag |
| U7-LESE44 | | acacaattgcggaagtgcgtctgtagcgagccagggaaggacatcaactccactttcgatga |
| | | gggtgagatcaaggtgccatttccacacccctccactgatatgtgaatcacaaagcacagttc |
| | | cttattcggttcgataaacaatattctaaaagactattaaaaccgctcgtttcttgagtttgtgacc |
| | | gcttgtaaaggctatgcaaatgagtcagtgctgattggctgaaaacagccaatcacagctcct |
| | | atgttgtta |
| SESE44 | | |
| SESE44 | 6 | TCAGTGGCTAACAGAAGCTGAACAGTTTCTCAGAA |
| target | | |
| sequence | | |
| SESE44 | 34 | UCAGUGGCUAACAGAAGCUGAACAGUUUCUCAGAA |
| target mRNA | | |
| sequence | | |
| SESE44 | 10 | TTCTGAGAAACTGTTCAGCTTCTGTTAGCCACTGA |
| antisense | | |
| sequence | | |
| SESE44 | 14 | UUCUGAGAAACUGUUCAGCUUCUGUUAGCCACUGA |
| antisense | | |
| mRNA | | |
| sequence | | |
| U7-SESE44 | 18 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaatt |
| | | ctgagaaactgttcagcttctgttagccactgaaatttttggagcaggttttctgacttcggtcgga |
| | | aaacccctcccaatttcactggtctacaatgaaagcaaaacagttctcttccccgctccccggt |
| | | gtgtgagaggggctttgatccttctctggtttcctaggaaacgcgtatgtg |
| Inverted or | 22 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatttcagtggctaacagaagctgaacagtttctcagaattg |
| U7-SESE44 | | cggaagtgcgtctgtagcgagccagggaaggacatcaactccactttcgatgagggtgaga |
| | | tcaaggtgccatttccacacccctccactgatatgtgaatcacaaagcacagttccttattcggtt |
| | | cgataaacaatattctaaaagactattaaaaccgctcgtttcttgagtttgtgaccgcttgtaaag |
| | | gctatgcaaatgagtcagtgctgattggctgaaaacagccaatcacagctcctatgttgtta |
| SD44 | | |
| SD44 Target | 7 | CAAATGGTATCTTAAGgtaag |
| sequence | | |
| 3′ part of Exon | | |
| 44 (upper | | |
| case) with | | |
| surrounding | | |
| intronic | | |
| sequence | | |
| (lower case) | | |
| SD44 target | 35 | CAAAUGGUAUCUUAAGguaag |
| mRNA | | |
| SD44 | 11 | cttacCTTAAGATACCATTTG |
| antisense | | |
| SD44 | 15 | cuuacCUUAAGAUACCAUUUG |
| antisense | | |
| mRNA | | |
| U7-SD44 | 19 | taacaacataggagctgtgattggctgttttcagccaatcagcactgactcatttgcatagccttt |
| | | acaagcggtcacaaactcaagaaacgagcggttttaatagtcttttagaatattgtttatcgaac |
| | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaac |
| | | ttaccttaagataccatttgaatttttggagcaggttttctgacttcggtcggaaaacccctcccaa |
| | | tttcactggtctacaatgaaagcaaaacagttctcttccccgctccccggtgtgtgagaggggc |
| | | tttgatccttctctggtttcctaggaaacgcgtatgtg |
| Inverted or | 23 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| U7-SD44 | | agcgagccagggaaggacatcaactccactttcgatgagggtgagatcaaggtgccatttcc |
| | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgtta |
| Additional U7 | | |
| Construct | | |
| Sequences | | |
| U7-4xSD44 | 24 | taacaacataggagctgtgattggctgttttcagccaatcagcactgactcatttgcatagccttt |
| (comprises 4 | | acaagcggtcacaaactcaagaaacgagcggttttaatagtcttttagaatattgtttatcgaac |
| copies of the | | cgaataaggaactgtgctttgtgattcacatatcagtggaggggtgtggaaatggcaccttgat |
| SD44 insert) | | ctcaccctcatcgaaagtggagttgatgtccttccctggctcgctacagacgcacttccgcaac |
| | | ttaccttaagataccatttgaatttttggagcaggttttctgacttcggtcggaaaacccctcccaa |
| | | tttcactggtctacaatgaaagcaaaacagttctcttccccgctccccggtgtgtgagaggggc |
| | | tttgatccttctctggtttcctaggaaacgcgtatgtggctagataacaacataggagctgtgatt |
| | | ggctgttttcagccaatcagcactgactcatttgcatagcctttacaagcggtcacaaactcaa |
| | | gaaacgagcggttttaatagtcttttagaatattgtttatcgaaccgaataaggaactgtgctttgt |
| | | gattcacatatcagtggaggggtgtggaaatggcaccttgatctcaccctcatcgaaagtgga |
| | | gttgatgtccttccctggctcgctacagacgcacttccgcaacttaccttaagataccatttgaatt |
| | | tttggagcaggttttctgacttcggtcggaaaacccctcccaatttcactggtctacaatgaaag |
| | | caaaacagttctcttccccgctccccggtgtgtgagaggggctttgatccttctctggtttcctagg |
| | | aaacgcgtatgtggctagataacaacataggagctgtgattggctgttttcagccaatcagca |
| | | ctgactcatttgcatagcctttacaagcggtcacaaactcaagaaacgagcggttttaatagtct |
| | | tttagaatattgtttatcgaaccgaataaggaactgtgctttgtgattcacatatcagtggagggg |
| | | tgtggaaatggcaccttgatctcaccctcatcgaaagtggagttgatgtccttccctggctcgct |
| | | acagacgcacttccgcaacttaccttaagataccatttgaatttttggagcaggttttctgacttcg |
| | | gtcggaaaacccctcccaatttcactggtctacaatgaaagcaaaacagttctcttccccgctc |
| | | cccggtgtgtgagaggggctttgatccttctctggtttcctaggaaacgcgtatgtggctagata |
| | | acaacataggagctgtgattggctgttttcagccaatcagcactgactcatttgcatagcctttac |
| | | aagcggtcacaaactcaagaaacgagcggttttaatagtcttttagaatattgtttatcgaaccg |
| | | aataaggaactgtgctttgtgattcacatatcagtggaggggtgtggaaatggcaccttgatctc |
| | | accctcatcgaaagtggagttgatgtccttccctggctcgctacagacgcacttccgcaactta |
| | | ccttaagataccatttgaatttttggagcaggttttctgacttcggtcggaaaacccctcccaattt |
| | | cactggtctacaatgaaagcaaaacagttctcttccccgctccccggtgtgtgagaggggcttt |
| | | gatccttctctggtttcctaggaaacgcgtatgtg |
| U7-SD44- | 25 | ctagaggctcgagaagatatcaactgcagcttctactgggcggttttatggacagcaagcga |
| stuffer | | accggaattgccagctggggcgccctctggtaaggttgggaagccctgcaaagtaaactgg |
| (comprises | | atggctttctcgccgccaaggatctgatggcgcaggggatcaagctctgatcaagagacagg |
| SD44 with | | atgaggatcgtttcgcgttcttgactcttcgcgatgtacgggccagatatacgcgttgacattgat |
| stuffer | | tattgactagttattaatagtaatcaattacggggtcattagttcatagcccatatatggagttccg |
| sequence) | | cctgcagggacgtcgacggatcgggagatctcccgatcccctatctgctccctgcttgtgtgttg |
| | | gaggtcgctgagtagtgcgcgagcaaaatttaagctacaacaaggcaaggcttgaccgac |
| | | aattgcatgaagaatctgcttagggttaggcgttttgcgctgcttcgcggcgcgccttttaaggc |
| | | agttattggtgcccttaaacgcctggtgctacgcctgaataagtgataataagcggatgaatgg |
| | | cagaaattcgccggatctttgtgaaggaaccttacttctgtggtgtgacataattggacaaacta |
| | | cctacagagatttaaagctctactagggtgggcgaagaactccagcatgagatccccgcgct |
| | | ggaggatcatccagccggcgtcccggaaaacgattccgaagcccaacctttcatagaagg |
| | | cggcggtggaatcgaaatctcgtgatggcaggttgggcgtcgcttggtcggtcatttcgaaccc |
| | | cagagtcccgctcagggcgcgccgggggggggggcgctgaggtctgcctcgtgaagaag |
| | | gtgttgctgactcataccaggcctgaatcgccccatcatccagccagaaagtgagggagcc |
| | | acggttgatgagagctttgttgtaggtggaccagtcctgcaggagcataaagtgtaaagcctg |
| | | gggtgcctaatgagtgagctaactcacattaattgcgttgcgctcactgcccgctttccagtcgg |
| | | gaaacctgtcgtgcccgcccagtctagctatcgccatgtaagcccactgcaagctacctgcttt |
| | | ctctttgcgcttgcgttttcccttgtccagatagcccagtagctgacattcatccggggtcagcac |
| | | cgtttctgcggactggctttctacgtgtctggttcgaggcgggatcagccaccgcggtggcggc |
| | | ctagagtcgacgaggaactgaaaaaccagaaagttaactggcctgtacggaagtgttacttc |
| | | tgctctaaaagctgcggaattgtacccgcggccgatccaccggtcgccaccagcggccatc |
| | | aagcacgttatcgataccgtcgactagagctcgctgatcagtggggggtggggtggggcag |
| | | gacagcaagggggaggattgggaagacaatagcagctgcagaagtttaaacgcatgtaa |
| | | caacataggagctgtgattggctgttttcagccaatcagcactgactcatttgcatagcctttaca |
| | | agcggtcacaaactcaagaaacgagcggttttaatagtcttttagaatattgtttatcgaaccga |
| | | ataaggaactgtgctttgtgattcacatatcagtggaggggtgtggaaatggcaccttgatctca |
| | | ccctcatcgaaagtggagttgatgtccttccctggctcgctacagacgcacttccgcaacttac |
| | | cttaagataccatttgaatttttggagcaggttttctgacttcggtcggaaaacccctcccaatttc |
| | | actggtctacaatgaaagcaaaacagttctcttccccgctccccggtgtgtgagaggggctttg |
| | | atccttctctggtttcctaggaaacgcgtatgtg |
| Inverted or | 26 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| U7-4xSD44 | | agcgagccagggaaggacatcaactccactttcgatgagggtgagatcaaggtgccatttcc |
| | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgttatctagccacatacgcgtt |
| | | tcctaggaaaccagagaaggatcaaagcccctctcacacaccggggagcggggaagag |
| | | aactgttttgctttcattgtagaccagtgaaattgggaggggttttccgaccgaagtcagaaaac |
| | | ctgctccaaaaattcaaatggtatcttaaggtaagttgcggaagtgcgtctgtagcgagccag |
| | | ggaaggacatcaactccactttcgatgagggtgagatcaaggtgccatttccacacccctcc |
| | | actgatatgtgaatcacaaagcacagttccttattcggttcgataaacaatattctaaaagacta |
| | | ttaaaaccgctcgtttcttgagtttgtgaccgcttgtaaaggctatgcaaatgagtcagtgctgaft |
| | | ggctgaaaacagccaatcacagctcctatgttgttatctagccacatacgcgtttcctaggaaa |
| | | ccagagaaggatcaaagcccctctcacacaccggggagcggggaagagaactgttttgctt |
| | | tcattgtagaccagtgaaattgggaggggttttccgaccgaagtcagaaaacctgctccaaa |
| | | aattcaaatggtatcttaaggtaagttgcggaagtgcgtctgtagcgagccagggaaggaca |
| | | tcaactccactttcgatgagggtgagatcaaggtgccatttccacacccctccactgatatgtg |
| | | aatcacaaagcacagttccttattcggttcgataaacaatattctaaaagactattaaaaccgc |
| | | tcgtttcttgagtttgtgaccgcttgtaaaggctatgcaaatgagtcagtgctgattggctgaaaa |
| | | cagccaatcacagctcctatgttgttatctagccacatacgcgtttcctaggaaaccagagaa |
| | | ggatcaaagcccctctcacacaccggggagcggggaagagaactgttttgctttcattgtaga |
| | | ccagtgaaattgggaggggttttccgaccgaagtcagaaaacctgctccaaaaattcaaatg |
| | | gtatcttaaggtaagttgcggaagtgcgtctgtagcgagccagggaaggacatcaactccac |
| | | tttcgatgagggtgagatcaaggtgccatttccacacccctccactgatatgtgaatcacaaag |
| | | cacagttccttattcggttcgataaacaatattctaaaagactattaaaaccgctcgtttcttgagt |
| | | ttgtgaccgcttgtaaaggctatgcaaatgagtcagtgctgattggctgaaaacagccaatca |
| | | cagctcctatgttgtta |
| Inverted or | 27 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaattcaaatggtatcttaaggtaagttgcggaagtgcgtctgt |
| U7-SD44- | | agcgagccagggaaggacatcaactccactttcgatgagggtgagatcaaggtgccatttcc |
| stuffer | | acacccctccactgatatgtgaatcacaaagcacagttccttattcggttcgataaacaatattc |
| | | taaaagactattaaaaccgctcgtttcttgagtttgtgaccgcttgtaaaggctatgcaaatgagt |
| | | cagtgctgattggctgaaaacagccaatcacagctcctatgttgttacatgcgtttaaacttctgc |
| | | agctgctattgtcttcccaatcctcccccttgctgtcctgccccaccccaccccccactgatcag |
| | | cgagctctagtcgacggtatcgataacgtgcttgatggccgctggtggcgaccggtggatcgg |
| | | ccgcgggtacaattccgcagcttttagagcagaagtaacacttccgtacaggccagttaacttt |
| | | ctggtttttcagttcctcgtcgactctaggccgccaccgcggtggctgatcccgcctcgaacca |
| | | gacacgtagaaagccagtccgcagaaacggtgctgaccccggatgaatgtcagctactgg |
| | | gctatctggacaagggaaaacgcaagcgcaaagagaaagcaggtagcttgcagtgggctt |
| | | acatggcgatagctagactgggcgggcacgacaggtttcccgactggaaagcgggcagtg |
| | | agcgcaacgcaattaatgtgagttagctcactcattaggcaccccaggctttacactttatgctc |
| | | ctgcaggactggtccacctacaacaaagctctcatcaaccgtggctccctcactttctggctgg |
| | | atgatggggcgattcaggcctggtatgagtcagcaacaccttcttcacgaggcagacctcag |
| | | cgcccccccccccggcgcgccctgagcgggactctggggttcgaaatgaccgaccaagcg |
| | | acgcccaacctgccatcacgagatttcgattccaccgccgccttctatgaaaggttgggcttcg |
| | | gaatcgttttccgggacgccggctggatgatcctccagcgcggggatctcatgctggagttctt |
| | | cgcccaccctagtagagctttaaatctctgtaggtagtttgtccaattatgtcacaccacagaag |
| | | taaggttccttcacaaagatccggcgaatttctgccattcatccgcttattatcacttattcaggcg |
| | | tagcaccaggcgtttaagggcaccaataactgccttaaaaggcgcgccgcgaagcagcgc |
| | | aaaacgcctaaccctaagcagattcttcatgcaattgtcggtcaagccttgccttgttgtagctta |
| | | aattttgctcgcgcactactcagcgacctccaacacacaagcagggagcagataggggatc |
| | | gggagatctcccgatccgtcgacgtccctgcaggcggaactccatatatgggctatgaacta |
| | | atgaccccgtaattgattactattaataactagtcaataatcaatgtcaacgcgtatatctggccc |
| | | gtacatcgcgaagagtcaagaacgcgaaacgatcctcatcctgtctcttgatcagagcttgat |
| | | cccctgcgccatcagatccttggcggcgagaaagccatccagtttactttgcagggcttccca |
| | | accttaccagagggcgccccagctggcaattccggttcgcttgctgtccataaaaccgccca |
| | | gtagaagctgcagttgatatcttctcgagcctctag |
| U7 sequences | | |
| U7 sequence | 28 | taacaacataggagctgtgattggctgttttcagccaatcagcactgactcatttgcatagccttt |
| (surrounding | | acaagcggtcacaaactcaagaaacgagcggttttaatagtcttttagaatattgtttatcgaac |
| insert) | | cgaataaggaactgtgctttgtgattcacatatcagtggaggggtgtggaaatggcaccttgat |
| | | ctcaccctcatcgaaagtggagttgatgtccttccctggctcgctacagacgcacttccgcaa- |
| | | ANTISENSE- |
| | | aatttttggagcaggttttctgacttcggtcggaaaacccctcccaatttcactggtctacaatga |
| | | aagcaaaacagttctcttccccgctccccggtgtgtgagaggggctttgatccttctctggtttcct |
| | | aggaaacgcgtatgtg |
| Inverted or | 29 | cacatacgcgtttcctaggaaaccagagaaggatcaaagcccctctcacacaccggggag |
| reverse | | cggggaagagaactgttttgctttcattgtagaccagtgaaattgggaggggttttccgaccga |
| complement | | agtcagaaaacctgctccaaaaatt-TARGET- |
| U7 sequence | | ttgcggaagtgcgtctgtagcgagccagggaaggacatcaactccactttcgatgagggtga |
| (surrounding | | gatcaaggtgccatttccacacccctccactgatatgtgaatcacaaagcacagttccttattcg |
| insert) | | gttcgataaacaatattctaaaagactattaaaaccgctcgtttcttgagtttgtgaccgcttgtaa |
| | | aggctatgcaaatgagtcagtgctgattggctgaaaacagccaatcacagctcctatgttgtta |

### Example 2

### Materials and Methods used in the Experiments

### Creation of Cell Lines

Skin biopsies were obtained from three patients that suffered from either an exon 45 deletion, an exon 44 duplication, or an exon 45-56 deletion. These skin biopsies were developed into three cell lines by infection using lentiviral vectors for both hTERT (to immortalize the cells) and MyoD (which forces transdifferentiation of the cells into myotubes) delivery to the fibroblasts to create myogenic fibroblasts (FibroMyoD) which express dystrophin. The FibroMyoD were infected with various rAAV preparations as described herein. 2.5e11 viral genome per 10 cm dishes were used. Four to eight days later, cells were collected and RNA and protein extractions were carried out.

### The hDMD/mdx del45 Mouse Model

The hDMD/mdx del45 mouse model (also referred to herein as the “hDMDdel45 mdx” model or “hDMD/del45 mdx” model) was obtained from Dr. Melissa Spencer [Young et al., J. Neuromuscul. Dis. 2017; 4(2): 139-145 (2017)]. This mouse contains the human version of the DMD gene but it contains a deletion of exon 45 of the human DMD gene in the hDMD mice resulting in an out of frame transcript. This mouse also contains a stop mutation in the murine DMD gene. Altogether, these two mutations lead to no human or murine dystrophin expression in this mouse model. Because the hDMD/mdx del45 mouse lacks both mouse and human dystrophin, the mouse presents with a dystrophic muscle pathology in multiple muscles across the body. This mouse model is used in various experiments described herein.

### RNA Extraction

RNA extraction was carried out on the cell pellet after centrifugation of the cells. Pellets were rinsed and 1 ml of TRIzol (Life Technologies) was added. Cell lysate was homogenized by pipetting and then it was incubated for 5 min at RT. Cell lysate was transferred into a 1.5 ml tube and 0.2 ml of chloroform was added per 1 ml of TRIzol. The lysate/TRIzol/chloroform mixture was shaken manually for 15 s. The mixture was then incubated for 2-3 min at RT and centrifuged for 15 min at 12,000 g (+4° C.). The aqueous phase (i.e., the upper one) was collected and transferred into a new tube. 0.5 ml of isopropanol (per ml of TRIzol) was added and allowed to stand for 10 min at RT. Supernatant was then removed after centrifugation at 12,000 g for 10 min at 4° C. and the pellet was washed with 1 ml of 75% EtOH (per ml of TRIzol). After centrifugation (7,500 g for 5 min at 4° C.), the pellet was air dried and the RNA was resuspended into RNAse free water for 10 min at 60° C.

### Reverse Transcription and PCR Amplification

This protocol is based on the manufacturer optimized protocol (Maxima Reverse Transcriptase, (Thermo Fisher Scientific). 1 μg of RNA was converted into cDNA. Two PCR primers were used for amplification (i.e., Fw: CTCCTGACCTCTGTGCTAAG (SEQ ID NO: 30); Rv: ATCTGCTTCCTCCAACCATAAAAC (SEQ ID NO: 31)). PCR amplification with an annealing temperature of 60° C.) was performed using the PCR Master Mix system (Thermo Fisher Scientific).

### Protein Extraction and Western Blotting

Mouse muscles lysates were prepared using lysis buffer (150 mM Tris-NaCl, 1% NP-40, digitonin (Sigma) and protease and phosphatases inhibitors (1860932, Thermo Inc.)). Lysates in buffer were incubated for one hour on ice. The lysate in buffer was then centrifuged at 14000 g for 20 min. Supernatant was collected. Protein quantification was performed using BCA protein assay kit (Pierce®). The supernatant was then mixed with a classic SDS-Page buffer and boiled 5 min at 100° C. 150 μg of each protein sample is run on a precast 3-8% Tris-Acetate gel (NuPage, Life Science) for 16 h at 80V (4° C.). Gels were transferred on a nitrocellulose membrane overnight at 300 mA.

Rabbit polyclonal antibodies against the C-terminal end of dystrophin were used (1:250, PA1-21011, Thermo Fisher Scientific; or 1:400, 15277, Abcam). Alpha-actinin (1:5000, A-7811, Sigma) was used as a loading control. After 1 hour incubation at RT, the membrane was washed (5×5 min with 0.1% Tween in TBS, TBST) and was exposed to the secondary antibodies (60 min at RT) at 1:1000 dilution. All antibodies were diluted in ½ Odyssey blocking buffer (Licor®) and ½ TBST. An anti-mouse IgG (H+L) (IRDye® 680CW Conjugate) and an anti-rabbit IgG (H+L) (IRDye® 800CW Conjugate) (Licor®) was used at 1:1000 dilution. 5×5 min with 0.1% Tween in TBS washes were performed followed by a ddH₂O soaking. The two simultaneous IRDye® signals were scanned using the LI-COR Odyssey® NIR. For muscle sections, immunoblotting was carried out for each muscle.

### Immunohistochemistry

Frozen muscles were cut at 8-10 microns and sections were air-dried before staining for 30 min. Sections were rehydrated in PBS and were incubated for 1 hour with normal goat serum (1:20) followed, only for mice sections, by a two hour incubation with an anti-mouse IgG unconjugated fab fragment at room temperature. The primary antibodies were left on overnight: Dystrophin (1:250, PA1-21011, Thermo Fisher Scientific). After washes, sections were incubated with the appropriate secondary antibody, i.e., Alexa Fluor 488 or 568-conjugated for 1 h (LifeScience). Slides were covered in Fluoromount plus DAPI (Vector Labs). Observations were realized using Olympus BX61. Acquisitions were taken using a DP controller (Olympus).

### Example 3

### In Vitro Transfection and Expression of rAAV Constructs that Target Exon 44 (AAV1.U7Δex44)

Skin biopsies were obtained from three patients that suffered from either an exon 45 deletion, an exon 44 duplication, or an exon 45-56 deletion. These skin biopsies were developed into three cell lines by infection using lentiviral vectors for both hTERT (to immortalize the cells) and MyoD (which forces transdifferentiation of the cells into myotubes) delivery to the fibroblasts to create myogenic fibroblasts (FibroMyoD) which express dystrophin. The FibroMyoD were infected with four different rAAV preparations.

Four different sequences [i.e., SEQ ID NOs: 4-7 (see Table 2), present in exon 44 or in exon 44 and the intronic sequence surrounding exon 4] were selected for targeting. U7snRNA constructs were designed to comprise each of SEQ ID NOs: 8-11 designed to bind to the target sequence. Each of the U7snRNA constructs (i.e., SEQ ID NOs: 16-25) was cloned into AAV1 to assess exon-skipping efficiency in myoblasts generated from those above described FibroMyoD.

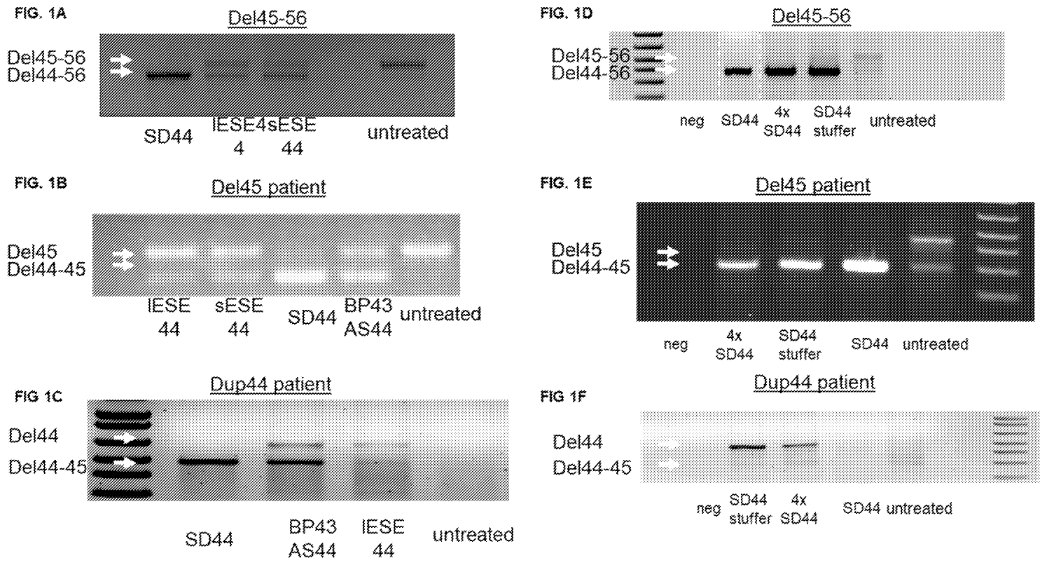

2.5e11 viral genome per 10 cm dishes were used. Four to eight days later, cells were collected and RNA and protein extractions were carried out. RT-PCR experiments were conducted in triplicate to observe exon skipping. All four AAV1.U7-antisense (i.e., AAV comprising each of SEQ ID NOs: 20-23) were able to mediate almost 100% of exon 44 skipping (FIG. 1A-C). Likewise, three AAV1.U7-antisense (i.e., AAV comprising each of SEQ ID NOs: 23, 26, and 27) were able to mediate almost 100% of exon 44 skipping (FIG. 1D-F).

Although efficient skipping of exon 4 was already demonstrated by constructs comprising BP43AS44, LESE44, SESE44, and SD44, four copies of SD44, i.e., U7.SD44, were cloned into the single self-complementary (sc) AAV1 vector (termed “U7-4xSD44”). In addition, because the exon skipping mediated by U7.SD44 was already so efficient, a construct carrying only one copy of U7.SD44 and an added stuffer sequence, i.e., random non-coding DNA, also was created.

2.5e11 viral genome per 10 cm dishes were used. Four to eight days later, cells were collected and RNA and protein extractions were carried out. RT-PCR experiments were conducted in triplicate to observe exon skipping. Three AAV1.U7-antisense (i.e., AAV comprising each of SEQ ID NOs: 23, 26, and 27) were used. AAV comprising each of SEQ ID NOs: 26 (4xSD44) and 27 (SD44-stuffer) were able to mediate almost 100% of exon 44 skipping (FIG. 1D-F). AAV1.U7-SD44 (AAV comprising SEQ ID NO: 23) was used as a positive control in this experiment.

### Example 4

### Intramuscular Delivery of rAAV Comprising U7-snRNAs Inducing Exon 44 Skipping (AAV9.U7Δex44) Results in Increased Dystrophin Expression

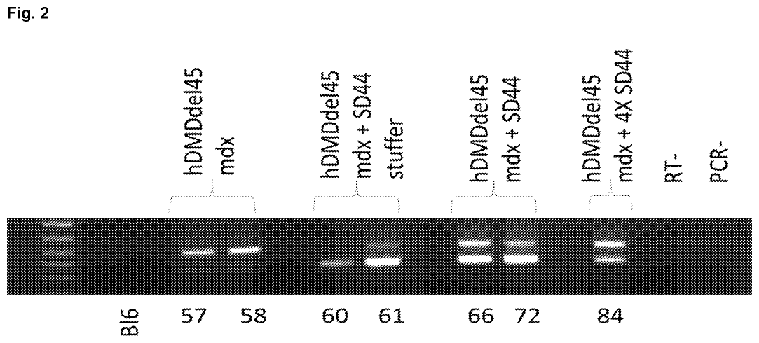

Six 2-month old hDMD/mdx del45 mice were injected with AAV1.U7-SD44 (AAV comprising SEQ ID NO: 23), AAV1.U7-SD44-stuffer (AAV comprising SEQ ID NO: 27) and AAV1.U7-4xSD44 (AAV comprising SEQ ID NO: 26), at 2.5 e11 AAV1 viral particles into each tibialis anterior (TA) muscle. Experiments were performed in each TA of two mice (n=4 TA muscles per construct). One month after viral injection, muscles were extracted from the 3-month old mice and exon skipping efficiency was determined by measuring human dystrophin expression by RT-PCR (FIG. 2). FIG. 2 shows the efficient skipping of human DMD exon 44 in the tibialis anterior (TA) muscle one month after injection with the three different rAAV viral vectors set forth above. These RT-PCR results demonstrated absence of exon skipping in mice #57 and #58 (untreated mice); efficient exon skipping in mice #60 and #61 (mice injected with U7-SD44-stuffer, i.e., AAV comprising SEQ ID NO: 27); efficient exon skipping in mice #66 and #72 (mice injected with U7-SD44, i.e., AAV comprising SEQ ID NO: 23); and efficient exon skipping in mouse #84 (mouse injected with U7-4xSD44, i.e., AAV comprising SEQ ID NO: 26). Black 6 (Bl6) is a wild-type mouse that does not contain the human DMD gene and, therefore, is a negative control for human DMD.

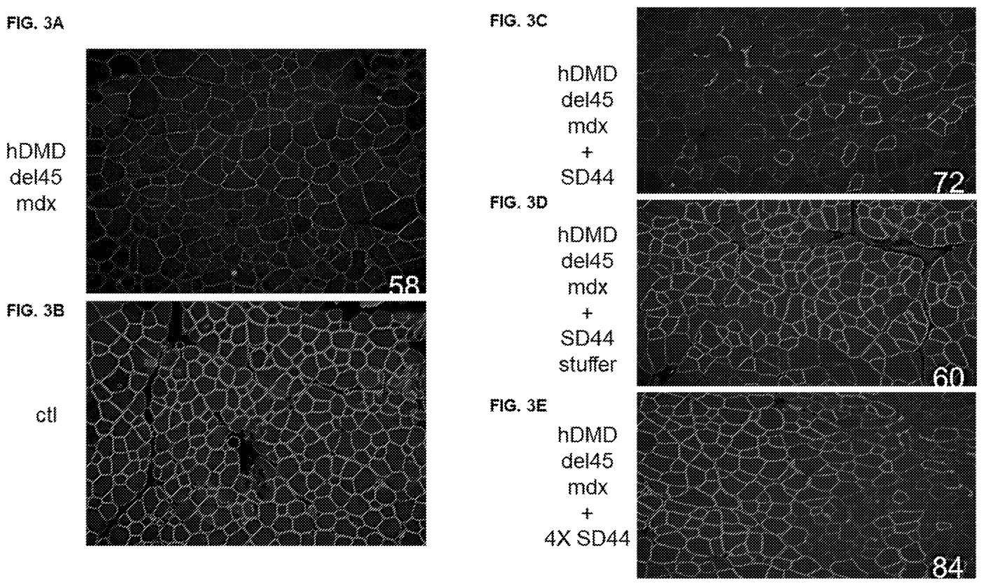

Dystrophin expression was confirmed by immunofluorescence (FIG. 3A-E). FIG. 3A-E shows the immunofluorescent expression of human dystrophin in the tibialis anterior (TA) muscle of 2-month old hDMD/mdx del45 mice one month after injection with the three different rAAV viral vectors. Experiments were performed in each TA of two mice (n=4 TA muscles per construct).

These immunofluorescence experimental results were obtained from #58 (untreated hDMD/mdx del45 mouse; FIG. 3A); from Black 6 (Bl6) control mouse (FIG. 3B), i.e., the Bl6 mouse that does not contain the human DMD gene; however, the antibody used in this immunofluorescence experiment recognizes both human and mouse dystrophin; from mouse #72 (mouse injected with U7.SD44; FIG. 3C); from mouse #60 (mouse injected with U7-SD44stuffer; FIG. 3D); and from mouse #84 (mouse injected with U7-4xSD44) (FIG. 3E).

After one month, immunostaining of muscle indicates that dystrophin was expressed after viral infection with all three rAAV vectors, with the SD44-stuffer vector (mice injected with U7-SD44-stuffer, i.e., AAV comprising SEQ ID NO: 27; FIG. 3D) and the 4x-SD44 vector (mice injected with U7-4xSD44, i.e., AAV comprising SEQ ID NO: 26; FIG. 3E) appearing to result in the greatest levels of dystrophin expression in the muscle.

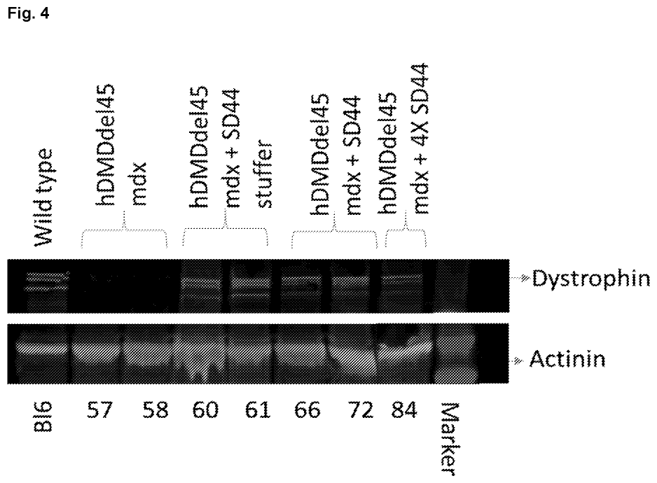

Dystrophin expression was confirmed by Western blot analysis (FIG. 4). FIG. 4 shows Western blot expression of human dystrophin in the tibialis anterior (TA) muscle of hDMD/mdx del45 mice one month after injection with the three different rAAV viral vectors. Experiments were performed in each TA of two mice (n=4 TA muscles per construct). After one month, Western blots result show that dystrophin was expressed after infection with all three rAAV viral vectors, with the SD44-stuffer vector appearing to result in the greatest level of dystrophin expression in the muscle. These Western blot results were obtained from mice #57 and #58 (untreated mice); from mice #60 and #61 (mice injected with U7.SD44-stuffer, i.e., AAV comprising SEQ ID NO: 27); from mice #66 and #72 (mice injected with U7.SD44, i.e., AAV comprising SEQ ID NO: 23) and from mouse #84 (mouse injected with U7.4xSD44, i.e., AAV comprising SEQ ID NO: 26). Dystrophin is expressed by the Bl6 control since the antibody used in this Western blot recognizes both human and mouse dystrophin.

Thus, the delivery of the AAV.U7snRNA-antisense in all three rAAV vectors comprising U7.SD44 (AAV comprising SEQ ID NO: 23), U7.4xSD44 (AAV comprising SEQ ID NO: 26), and U7.SD44-stuffer (AAV comprising SEQ ID NO: 27) induced dystrophin expression by targeting exon 44, including targeting intronic sequence adjacent to exon 44. While all constructs mediated robust exon skipping leading to strong dystrophin expression, the rAAV comprising the SD44-stuffer construct and the 4x-SD44 construct ((FIG. 3D-E and FIG. 4) appeared to be more efficient than the others in these experiments.

### Example 5

### Systemic Delivery of rAAV Comprising U7-snRNAs Inducing Exon 44 Skipping (AAV9.U7Δex44) Results in Increased Dystrophin Expression

Ten hDMDdel45/mdx mice (two month old) are injected with AAV9.U7-SD4-stuffer or AAV9.U7-4X-SD44 (SEQ ID NOs: 27 and 26, respectively, cloned into AAV9) with various doses ranging from 3e13 vg/kg to 2e14 vg/kg into the temporal vein (i.e., neonatal mice) or the tail vein (i.e., 2-month old mice). Mice transduced with these viral vectors are collected at one, three, or six months post-injection. Exon skipping efficiency is determined by measuring dystrophin expression by RT-PCR, immunofluorescence, and by Western blot analysis using protocols described herein above.

While the present disclosure has been described in terms of specific embodiments, it is understood that variations and modifications will occur to those skilled in the art. Accordingly, only such limitations as appear in the claims should be placed on the disclosure.

All documents referred to in this application are hereby incorporated by reference in their entirety with particular attention to the content for which they are referred.

What is claimed is:

1. A nucleic acid molecule that binds or is complementary to a polynucleotide encoding exon 44 of the DMD gene, wherein the polynucleotide encoding exon 44 comprises or consists of the nucleotide sequence set out in SEQ ID NO: 1 or 2 or encodes the amino acid sequence set out in SEQ ID NO: 3.

2. The nucleic acid molecule of claim 1 that binds or is complementary to at least one of the nucleotide sequences set out in SEQ ID NO: 4, 5, 6, 7, 32, 33, 34, or 35.

3. The nucleic acid molecule of claim 1 or 2 comprising or consisting of a nucleotide sequence having at least 80%, at least 85%, at least 90%, at least 95%, at least 96%, at least 97%, at least 98%, or at least 99% identity to the nucleotide sequence set out in SEQ ID NO: 4, 5, 6, 7, 8, 9, 10, 11, 12, 13, 14, 15, 32, 33, 34, or 35.

4. The nucleic acid molecule of any one of claims 1-3 comprising or consisting of a nucleotide sequence having at least 80%, at least 85%, at least 90%, at least 95%, at least 96%, at least 97%, at least 98%, or at least 99% identity to the nucleotide sequence set out in SEQ ID NO: 16, 17, 18, 19, 20, 21, 22, 23, 24, 25, 26, 27, or 28.

5. The nucleic acid molecule of claim 1, 2, or 3 comprising or consisting of the nucleotide sequence set out in SEQ ID NO: 4, 5, 6, 7, 8, 9, 10, 11, 12, 13, 14, 15, 32, 33, 34, or 35.

6. The nucleic acid molecule of any one of claims 1-4 comprising or consisting of the nucleotide sequence set out in SEQ ID NO: 16, 17, 18, 19, 20, 21, 22, 23, 24, 25, 26, 27, or 28.

7. A recombinant adeno-associated virus (rAAV) comprising a genome comprising at least one of the nucleic acid molecules of any one of claims 1-6.

8. The rAAV of claim 7 wherein the genome is a self-complementary genome or a single-stranded genome.

9. The rAAV of claim 7 or 8 wherein the rAAV is rAAV-1, rAAV-2, rAAV-3, rAAV-4, rAAV-5, rAAV-6, rAAV-7, rAAV-8, rAAV-9, rAAV-10, rAAV-11, rAAV-12, rAAV-13, rAAV-rh74, or rAAV-anc80.

10. The rAAV of claim of any one of claims 7-9 wherein the genome of the rAAV lacks AAV rep and cap DNA.

11. The rAAV of claim 10 further comprising an AAV-1 capsid, an AAV-2 capsid, an AAV-3 capsid, an AAV-4 capsid, an AAV-5 capsid, an AAV-6 capsid, an AAV-7 capsid, an AAV-8 capsid, an AAV-9 capsid, an AAV-10 capsid, an AAV-11 capsid, an AAV-12 capsid, an AAV-13 capsid, an AAV-rh74 capsid, or an AAV-anc80 capsid.

12. A method for inducing skipping of exon 44 of the DMD gene in a cell, the method comprising providing the cell with the nucleic acid molecule of any one of claims 1-6.

13. A method for inducing skipping of exon 44 of the DMD gene in a cell, the method comprising providing the cell with the rAAV of any one of claims 7-11.

14. A method for treating, ameliorating, and/or preventing a muscular dystrophy in a subject with a mutation amenable to skipping exon 44 of the DMD gene (DMD exon 44) comprising administering to the subject at least one of the nucleic acid molecules of any one of claims 1-6.

15. A method for treating, ameliorating, and/or preventing a muscular dystrophy in a subject with a mutation amenable to skipping exon 44 of the DMD gene (DMD exon 44) comprising administering to the subject at least one of the rAAV of any one of claims 7-11.

16. The method of claim 14 or 15, wherein the mutation is any mutation involving, surrounding, or affecting DMD exon 44.

17. The method of claim 16, wherein the mutation is a duplication of DMD exon 44, a deletion of exon 43 or 45, or a deletion of exons 45-56.

18. The method of any one of claims 14-17, wherein the administering results in increased expression of dystrophin protein in the subject.

19. The method of any one of claims 14-17, wherein the administering inhibits the progression of dystrophic pathology in the subject.

20. The method of any one of claims 14-17, wherein the administering improves muscle function in the subject.

21. The method of claim 20 wherein the improvement in muscle function is an improvement in muscle strength.

22. The method of claim 20 wherein the improvement in muscle function is an improvement in stability in standing and walking.

23. Use of at least one nucleic acid molecule of any one of claims 1-6 in treating, ameliorating, and/or preventing a muscular dystrophy in a subject with a mutation amenable to skipping exon 44 of the DMD gene (DMD exon 44).

24. Use of at least one rAAV of any one of claims 7-11 in treating, ameliorating, and/or preventing a muscular dystrophy in a subject with a mutation amenable to skipping exon 44 of the DMD gene (DMD exon 44).


SEQUENCE LISTING (35 sequences)

SEQ ID NO: 1 - DNA, 300 bp, Artificial Sequence
  Synthetic Polynucleotide
ttgtcagtataaccaaaaaatatacgctatatctctataatctgttttacataatccatctatttttcttgatccatatgcttttacctgcaggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggtaagtctttgatttgttttttcgaaattgtatttatcttcagcacatctggactcttt

SEQ ID NO: 2 - DNA, 148 bp, Artificial Sequence
  Synthetic Polynucleotide
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 3 - protein, 49 aa, Artificial Sequence
  Synthetic Polypeptide
RFDRSVEKWRRFHYDIKIFNQWLTEAEQFLRKTQIPENWEHAKYKWYLK

SEQ ID NO: 4 - DNA, 43 bp, Artificial Sequence
  Synthetic Polynucleotide
tttcttgatccatatgcttttacctgcaggcgatttgacagat

SEQ ID NO: 5 - DNA, 43 bp, Artificial Sequence
  Synthetic Polynucleotide
tcagtggctaacagaagctgaacagtttctcagaaagacacaa

SEQ ID NO: 6 - DNA, 35 bp, Artificial Sequence
  Synthetic Polynucleotide
tcagtggctaacagaagctgaacagtttctcagaa

SEQ ID NO: 7 - DNA, 21 bp, Artificial Sequence
  Synthetic Polynucleotide
caaatggtatcttaaggtaag

SEQ ID NO: 8 - DNA, 43 bp, Artificial Sequence
  Synthetic Polynucleotide
atctgtcaaatcgcctgcaggtaaaagcatatggatcaagaaa

SEQ ID NO: 9 - DNA, 43 bp, Artificial Sequence
  Synthetic Polynucleotide
ttgtgtctttctgagaaactgttcagcttctgttagccactga

SEQ ID NO: 10 - DNA, 35 bp, Artificial Sequence
  Synthetic Polynucleotide
ttctgagaaactgttcagcttctgttagccactga

SEQ ID NO: 11 - DNA, 21 bp, Artificial Sequence
  Synthetic Polynucleotide
cttaccttaagataccatttg

SEQ ID NO: 12 - RNA, 43 nt, Artificial Sequence
  Synthetic Polynucleotide
aucugucaaaucgccugcagguaaaagcauauggaucaagaaa

SEQ ID NO: 13 - RNA, 43 nt, Artificial Sequence
  Synthetic Polynucleotide
uugugucuuucugagaaacuguucagcuucuguuagccacuga

SEQ ID NO: 14 - RNA, 35 nt, Artificial Sequence
  Synthetic Polynucleotide
uucugagaaacuguucagcuucuguuagccacuga

SEQ ID NO: 15 - RNA, 21 nt, Artificial Sequence
  Synthetic Polynucleotide
cuuaccuuaagauaccauuug

SEQ ID NO: 16 - DNA, 450 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaatctgtcaaatcgcctgcaggtaaaagcatatggatcaagaaaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 17 - DNA, 450 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaattgtgtctttctgagaaactgttcagcttctgttagccactgaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 18 - DNA, 442 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaattctgagaaactgttcagcttctgttagccactgaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 19 - DNA, 428 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 20 - DNA, 1730 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggctagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggctagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggctagataacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 21 - DNA, 1933 bp, Artificial Sequence
  Synthetic Polynucleotide
ctagaggctcgagaagatatcaactgcagcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctactagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttaccttaagataccatttgaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 22 - DNA, 450 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttttcttgatccatatgcttttacctgcaggcgatttgacagatttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 23 - DNA, 450 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttcagtggctaacagaagctgaacagtttctcagaaagacacaattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 24 - DNA, 442 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttcagtggctaacagaagctgaacagtttctcagaattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 25 - DNA, 428 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 26 - DNA, 1730 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 27 - DNA, 1933 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattcaaatggtatcttaaggtaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttacatgcgtttaaacttctgcagctgctattgtcttcccaatcctcccccttgctgtcctgccccaccccaccccccactgatcagcgagctctagtcgacggtatcgataacgtgcttgatggccgctggtggcgaccggtggatcggccgcgggtacaattccgcagcttttagagcagaagtaacacttccgtacaggccagttaactttctggtttttcagttcctcgtcgactctaggccgccaccgcggtggctgatcccgcctcgaaccagacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcgggcacgacaggtttcccgactggaaagcgggcagtgagcgcaacgcaattaatgtgagttagctcactcattaggcaccccaggctttacactttatgctcctgcaggactggtccacctacaacaaagctctcatcaaccgtggctccctcactttctggctggatgatggggcgattcaggcctggtatgagtcagcaacaccttcttcacgaggcagacctcagcgcccccccccccggcgcgccctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagtagagctttaaatctctgtaggtagtttgtccaattatgtcacaccacagaagtaaggttccttcacaaagatccggcgaatttctgccattcatccgcttattatcacttattcaggcgtagcaccaggcgtttaagggcaccaataactgccttaaaaggcgcgccgcgaagcagcgcaaaacgcctaaccctaagcagattcttcatgcaattgtcggtcaagccttgccttgttgtagcttaaattttgctcgcgcactactcagcgacctccaacacacaagcagggagcagataggggatcgggagatctcccgatccgtcgacgtccctgcaggcggaactccatatatgggctatgaactaatgaccccgtaattgattactattaataactagtcaataatcaatgtcaacgcgtatatctggcccgtacatcgcgaagagtcaagaacgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagctgcagttgatatcttctcgagcctctag

SEQ ID NO: 28 - DNA, 407 bp, Artificial Sequence
  Synthetic Polynucleotide
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 29 - DNA, 407 bp, Artificial Sequence
  Synthetic Polynucleotide
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 30 - DNA, 20 bp, Artificial Sequence
  Synthetic Polynucleotide
ctcctgacctctgtgctaag

SEQ ID NO: 31 - DNA, 24 bp, Artificial Sequence
  Synthetic Polynucleotide
atctgcttcctccaaccataaaac

SEQ ID NO: 32 - RNA, 43 nt, Artificial Sequence
  Synthetic Polynucleotide
uuucuugauccauaugcuuuuaccugcaggcgauuugacagau

SEQ ID NO: 33 - RNA, 43 nt, Artificial Sequence
  Synthetic Polynucleotide
ucaguggcuaacagaagcugaacaguuucucagaaagacacaa

SEQ ID NO: 34 - RNA, 35 nt, Artificial Sequence
  Synthetic Polynucleotide
ucaguggcuaacagaagcugaacaguuucucagaa

SEQ ID NO: 35 - RNA, 21 nt, Artificial Sequence
  Synthetic Polynucleotide
caaaugguaucuuaagguaag